(19) United States
(12) Patent Application Publication
(10) Pub. No.: US 2011/0152341 A1
(43) Pub. Date: Jun. 23, 2011

(54) CLEAVAGE OF B-AMYLOID PRECURSOR PROTEIN

(75) Inventors: Stephan Schilling, Halle/Saale (DE); Dagmar Schlenzig, Halle/Saale (DE); Hans-Ulrich Demuth, Halle/Saale (DE)

(73) Assignee: PROBIODRUG AG, Halle/Saale (DE)

(21) Appl. No.: 12/975,615

(22) Filed: Dec. 22, 2010

Related U.S. Application Data
(60) Provisional application No. 61/288,971, filed on Dec. 22, 2009.

(51) Int. Cl. A61K 31/40; C12P 21/00; C12Q 1/37; A61K 31/381; A61P 35/00; A61P 25/18; A61P 35/04; A61P 17/06; A61P 25/00; A61P 25/28

(52) U.S. Cl. 514/423; 435/68.1; 435/23; 514/445

(57) ABSTRACT

Methods and means for the identification of meprin-α and meprin-β as novel β-secretases and antagonists thereof for use in the treatment of amyloidosis.

### CROSS-REFERENCE TO RELATED APPLICATIONS

This application claims priority to U.S. Provisional Application Ser. No. 61/288,971, filed on Dec. 22, 2009, which is incorporated herein by reference in its entirety.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present invention concerns methods and means for the identification of novel β-secretases obtained by the proteolytic processing of the β-amyloid precursor protein, APP, antagonists thereof and their use in the treatment of amyloidosis.

### BACKGROUND OF THE INVENTION

Amyloidosis is not a single disease entity but rather a diverse group of progressive disease processes characterized by extracellular tissue deposits of a waxy, starch-like protein called amyloid, which accumulates in one or more organs or body systems. As the amyloid deposits accumulate, they begin to interfere with the normal function of the organ or body system. There are at least 15 different types of amyloidosis. The major forms are primary amyloidosis without known antecedent, secondary amyloidosis following some other condition, and hereditary amyloidosis.

Secondary amyloidosis occurs during chronic infection or inflammatory disease, such as tuberculosis, a bacterial infection called familial Mediterranean fever, bone infections (osteomyelitis), rheumatoid arthritis, inflammation of the small intestine (granulomatous ileitis), Hodgkin's disease and leprosy.

Amyloid deposits include amyloid P (pentagonal) component (AP), a glycoprotein related to normal serum amyloid P (SAP), and sulphated glycosaminoglycans (GAG), complex carbohydrates of connective tissue. Amyloid protein fibrils, which account for about 90% of the amyloid material, comprise one of several different types of proteins. These proteins are capable of folding into so-called “beta-pleated” sheet fibrils, a unique protein configuration which exhibits binding sites for Congo red resulting in the unique staining properties of the amyloid protein.

Many diseases of aging are based on or associated with amyloid-like proteins and are characterized, in part, by the buildup of extracellular deposits of amyloid or amyloid-like material that contribute to the pathogenesis, as well as the progression of the disease. These diseases include, but are not limited to, neurological disorders such as mild cognitive impairment (MCI), Alzheimer's disease (AD), like for instance sporadic Alzheimer's disease (SAD) or Familial Alzheimer's dementias (FAD) like Familial British Dementia (FBD) and Familial Danish Dementia (FDD), neurodegeneration in Down Syndrome, Lewy body dementia, hereditary cerebral hemorrhage with amyloidosis (Dutch type); the Guam Parkinson-Dementia complex. Other diseases which are based on or associated with amyloid-like proteins are progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, and others, including macular degeneration.

Although pathogenesis of these diseases may be diverse, their characteristic deposits often contain many shared molecular constituents. To a significant degree, this may be attributable to the local activation of pro-inflammatory pathways thereby leading to the concurrent deposition of activated complement components, acute phase reactants, immune modulators, and other inflammatory mediators (McGeer et al., Tohoku J Exp Med. 174(3): 269-277 (1994)).

A number of important neurological diseases, including Alzheimer's disease (AD), cerebral amyloid angiopathy (CAA), and prion-mediated diseases are characterized by the deposition of aggregated proteins, referred to amyloid, in the central nervous system (CNS) (for reviews, see Glenner et al., J. Neurol. Sci. 94: 1-28 (1989); Haan et al., Clin. Neurol. Neurosurg. 92 (4): 305-310 (1990)). These highly insoluble aggregates are composed of nonbranching, fibrilla proteins with the common characteristic of β-pleated sheet conformation. In the CNS, amyloid can be present in cerebral and meningeal blood vessels (cerebrovascular deposits) and in the brain parenchyma (plaques). Neuropathological studies in human and animal models indicate that cells proximal to amyloid deposits are disturbed in their normal functions (Mandybur, Acta Neuropathol. 78: 329-331 (1989); Kawai et al., Brain Res. 623: 142-146 (1993); Martin et al., Am. J. Pathol. 145: 1348-1381 (1994); Kalaria et al., Neuroreport 6: 477-480 (1995); Masliah et al., J. Neurosci. 16: 5795-5811 (1996); Selkoe, J. Biol. Chem. 271: 18295-18298 (1996); Hardy, Trends Neurosci 20: 154-159 (1997)).

AD and CAA share biochemical and neuropathological markers, but differ somewhat in the extent and location of amyloid deposits as well as in the symptoms exhibited by affected individuals. The neurodegenerative process of AD, the most common neurodegenerative disorder worldwide, is characterized by the progressive and irreversible deafferentation of the limbic system, association neocortex, and basal forebrain accompanied by neuritic plaque and tangle formation (for a review, see Terry et al., “Structural alteration in Alzheimer's disease,” In: Alzheimer's disease, Terry et al. Eds., 1994, pp. 179-196, Raven Press, New York). Dystrophic neurites, as well as reactive astocytes and microglia, are associated with these amyloid-associated neuritic plaques. Although the neuritic population in any given plaque is mixed, the plaques generally are composed of spherical neurites that contain synaptic proteins, APP (type 1), and fusiform neurites containing cytoskeletal proteins and paired helical filaments (PHF; type 11).

CAA patients display various vascular syndromes, of which the most documented is cerebral parenchymal hemorrhage. Cerebral parenchymal hemorrhage is the result of extensive amyloid deposition within cerebral vessels (Hardy, Trends Neurosci 20: 154-159 (1997); Haan et al., Clin. Neurol. Neurosurg. 92: 305-310 (1990); Terry et al., (1994) supra; Vinters, Stroke 18: 211-224 (1987); Itoh et al., J. Neurosurgical Sci. 116: 135 141 (1993); Yamada et al., J. Neural. Neruosurg. Psychiatry 56: 543-547 (1993); Greenberg et al., Neurology 43: 2073-2079 (1993); Levy et al., Science 248: 1124-1126 (1990)). In some familial CAA cases, dementia was noted before the onset of hemorrhages, suggesting the possibility that cerebrovascular amyloid deposits may also interfere with cognitive functions.

Both AD and CAA are characterized by the accumulation of senile plaques in the brains of the affected individuals. The main amyloid components is the amyloid β protein (Aβ), also referred to as amyloid β or β-amyloid peptide, derived from proteolytic processing of the β-amyloid precursor protein, (β-APP or simply APP). For review in connection with AD see, Selkoe, D. J. Nature 399: A23-A31 (1999). Aβ is produced by proteolytic cleavage of an integral membrane protein, termed the (β-amyloid precursor protein (βAPP).

The Aβ peptide, which is generated from APP by two putative secretases, is present at low levels in the normal CNS and blood. Two major variants, Aβ₁₋₄₀ and Aβ₁₋₄₂ are produced by alternative carboxy-terminal truncation of APP (Selkoe et al. (1988) Proc. Natl. Acad. Sci. USA 85: 7341-7345; Selkoe (1993) Trends Neurosci 16: 403-409).

Aβ₁₋₄₂ is the more fibrillogenic and more abundant of the two peptides in amyloid deposits of both AD and CAA. In addition to the amyloid deposits in AD cases described above, most AD cases are also associated with amyloid deposition in the vascular walls (Hardy (1997), supra; Haan et al. (1990), supra; Terry et al., (1994) supra; Vinters (1987), supra; Itoh, et al. (1993), supra; Yamada et al. (1993), supra; Greenberg et al. (1993), supra; Levy et al. (1990), supra). These vascular lesions are the hallmark of CAA, which can exist in the absence of AD.

Recently, accumulating evidence demonstrates involvement of N-terminal modified Aβ peptide variants in Alzheimer's disease. Aiming biopsies display a presence of Aβ 1-40 and Aβ 1-42 not only in the brain of Alzheimer's patients but also in senile plaques of unaffected individuals. However, N-terminal truncated and pyroGlu modified Aβ N3pE-40/Aβ N3pE-42 is almost exclusively engrained within plaques of Alzheimer's disease patients, making this Aβ variant an eligible diagnostic marker and a potential target for drug development.

The brains of Alzheimer's disease (AD) patients are morphologically characterized by the presence of neurofibrillary tangles and by deposits of Aβ peptides in neocortical brain structures (Selkoe, D. J. & Schenk, D. Alzheimer's disease: molecular understanding predicts amyloid-based therapeutics. Annu. Rev. Pharmacol. Toxicol. 43, 545-584 (2003)). Aβ peptides are liberated from the amyloid precursor protein (APP) after sequential cleavage by β- and γ-secretase. The γ-secretase cleavage results in the generation of Aβ 1-40 and Aβ 1-42 peptides, which differ in their C-termini and exhibit different potencies of aggregation, fibril formation and neurotoxicity (Shin, R. W. et al. Amyloid beta-protein (Abeta) 1-40 but not Abeta 1-42 contributes to the experimental formation of Alzheimer disease amyloid fibrils in rat brain. J. Neurosci. 17, 8187-8193 (1997); Iwatsubo, T. et al. Visualization of Abeta 42(43) and Abeta 40 in senile plaques with end-specific Abeta monoclonals: evidence that an initially deposited species is Abeta 42(43). Neuron 13, 45-53 (1994); Iwatsubo, T., Mann, D. M., Odaka, A., Suzuki, N. & Ihara, Y. Amyloid beta protein (Abeta) deposition: Abeta 42(43) precedes Abeta 40 in Down syndrome. Ann. Neurol. 37, 294-299 (1995); Hardy, J. A. & Higgins, G. A. Alzheimer's disease: the amyloid cascade hypothesis. Science 256, 184-185 (1992); Roβner, S., Ueberham, U., Schliebs, R., Perez-Polo, J. R. & Bigl, V. The regulation of amyloid precursor protein metabolism by cholinergic mechanisms and neurotrophin receptor signaling. Prog. Neurobiol. 56, 541-569 (1998)). In addition to C-terminal variability, N-terminally modified Aβ peptides are abundant (Saido, T. C. et al. Dominant and differential deposition of distinct beta-amyloid peptide species, A beta N3(pE), in senile plaques. Neuron 14, 457-466 (1995); Russo, C. et al. Presenilin-1 mutations in Alzheimer's disease. Nature 405, 531-532 (2000); Saido, T. C., Yamao, H., Iwatsubo, T. & Kawashima, S. Amino- and carboxyl-terminal heterogeneity of beta-amyloid peptides deposited in human brain. Neurosci. Lett. 215, 173-176 (1996)). It appears that a major proportion of the Aβ peptides undergoes N-terminal truncation by two amino acids, exposing a glutamate residue, which is subsequently cyclized into pyroglutamate (pE), resulting in Aβ3(pE)-42 peptides (Saido, T. C. et al. Dominant and differential deposition of distinct beta-amyloid peptide species, A beta N3(pE), in senile plaques. Neuron 14, 457-466 (1995); Saido, T. C., Yamao, H., Iwatsubo, T. & Kawashima, S Amino- and carboxyl-terminal heterogeneity of beta-amyloid peptides deposited in human brain. Neurosci. Lett. 215, 173-176 (1996)). Alternatively, pE may be formed following β′-cleavage by BACE1, resulting in Aβ N11(pE)-42 (Naslund, J. et al. Relative abundance of Alzheimer A beta amyloid peptide variants in Alzheimer disease and normal aging. Proc. Natl. Acad. Sci. U.S.A. 91, 8378-8382 (1994); Liu, K. et al. Characterization of Abeta11-40/42 peptide deposition in Alzheimer's disease and young Down's syndrome brains: implication of N-terminally truncated Abeta species in the pathogenesis of Alzheimer's disease. Acta Neuropathol. 112, 163-174 (2006)). In particular Aβ N3(pE)-42 has been shown to be a major constituent of Aβ deposits in sporadic and familial Alzheimer's disease (FAD) (Saido, T. C. et al. Dominant and differential deposition of distinct beta-amyloid peptide species, A beta N3(pE), in senile plaques. Neuron 14, 457-466 (1995); Miravalle, L. et al. Amino-terminally truncated Abeta peptide species are the main component of cotton wool plaques. Biochemistry 44, 10810-10821 (2005)).

The Aβ N3pE-42 peptides coexist with Aβ 1-40/1-42 peptides (Saido, T. C. et al. Dominant and differential deposition of distinct beta-amyloid peptide species, Abeta N3pE, in senile plaques. Neuron 14, 457-466 (1995); Saido, T. C., Yamao, H., Iwatsubo, T. & Kawashima, S. Amino- and carboxyl-terminal heterogeneity of beta-amyloid peptides deposited in human brain. Neurosci. Lett. 215, 173-176 (1996)), and, based on a number of observations, could play a prominent role in the pathogenesis of AD. For example, a particular neurotoxicity of Aβ N3pE-42 peptides has been outlined (Russo, C. et al. Pyroglutamate-modified amyloid beta-peptides—AbetaN3(pE)—strongly affect cultured neuron and astrocyte survival. J. Neurochem. 82, 1480-1489 (2002) and the pE-modification of N-truncated Aβ peptides confers resistance to degradation by most aminopeptidases as well as Aβ-degrading endopeptidases (Russo, C. et al. Pyroglutamate-modified amyloid beta-peptides—AbetaN3(pE)—strongly affect cultured neuron and astrocyte survival. J. Neurochem. 82, 1480-1489 (2002); Saido, T. C. Alzheimer's disease as proteolytic disorders: anabolism and catabolism of beta-amyloid. Neurobiol. Aging 19, S69-S75 (1998)). The cyclization of glutamic acid into pE leads to a loss of N-terminal charge resulting in accelerated aggregation of Aβ N3pE compared to the unmodified Aβ peptides (He, W. & Barrow, C. J. The Abeta 3-pyroglutamyl and 11-pyroglutamyl peptides found in senile plaque have greater beta-sheet forming and aggregation propensities in vitro than full-length A beta. Biochemistry 38, 10871-10877 (1999); Schilling, S. et al. On the seeding and oligomerization of pGlu-amyloid peptides (in vitro). Biochemistry 45, 12393-12399 (2006)). Thus, reduction of Aβ N3pE-42 formation should destabilize the peptides by making them more accessible to degradation and would, in turn, prevent the formation of higher molecular weight Aβ aggregates and enhance neuronal survival.

However, for a long time it was not known how the pE-modification of Aβ peptides occurs. Recently, it was discovered that glutaminyl cyclase (QC) is capable to catalyze Aβ N3pE-42 formation under mildly acidic conditions and that specific QC inhibitors prevent Aβ N3pE-42 generation in vitro (Schilling, S., Hoffmann, T., Manhart, S., Hoffmann, M. & Demuth, H.-U. Glutaminyl cyclases unfold glutamyl cyclase activity under mild acid conditions. FEBS Lett. 563, 191-196 (2004); Cynis, H. et al. Inhibition of glutaminyl cyclase alters pyroglutamate formation in mammalian cells. Biochim. Biophys. Acta 1764, 1618-1625 (2006)).

The precise mechanisms by which neuritic plaques are formed and the relationship of plaque formation to the AD-associated, and CAA-associated neurodegenerative processes are not well defined. However, evidence indicates that dysregulated expression and/or processing of APP gene products or derivatives of these gene products are involved in the pathophysiological process leading to neurodegeneration and plaque formation. For example, missense mutations in APP are tightly linked to autosomal dominant forms of AD (Hardy (1994) Clin. Geriatr. Med. 10: 239-247; Mann et al. (1992) Neurodegeneration 1: 201-215). The role of APP in neurodegenerative diseases is further implicated by the observation that persons with Down's syndrome who carry an additional copy of the human APP (hAPP) gene on their third chromosome 21 show an overexpression of hAPP (Goodison et al. (1993) J. Neuropathol. Exp. Neurol. 52: 192-198; Oyama, et al. (1994) J. Neurochem. 62: 1062-1066) as well as a prominent tendency to develop AD-type pathology early in life (Wisniewski et al. (1985) Ann. Neurol. 17: 278-282). Mutations in Aβ are linked to CAA associated with hereditary cerebral hemorrhage with amyloidosis (Dutch HCHWA) (Levy, et al. (1990), supra), in which amyloid deposits preferentially occur in the cerebrovascular wall with some occurrence of diffuse plaques (Maat-Schieman et al. (1994) Acta Neuropathol. 88: 371-8; Wartendorff et al. (1995) J. Neurol. Neurosurg. Psychiatry 58: 699-705). A number of hAPP point mutations that are tightly associated with the development of familial AD encode amino acid changes close to either side of the Aβ peptide (for a review, see, e.g., Lannfelt et al. (1994) Biochem. Soc. Trans. 22: 176-179; Clark et al. (1993) Arch. Neurol. 50: 1164-1172). Finally, in vitro studies indicate that aggregated Aβ can induce neurodegeneration (see, e.g., Pike et al. (1995) J. Neurochem. 64: 253-265).

APP is a glycosylated, single-membrane-spanning protein expressed in a wide variety of cells in many mammalian tissues. Examples of specific isotypes of APP which are currently known to exist in humans are the 695-amino acid polypeptide (APP695) described by Kang et al. (1987) Nature 325: 733-736, which is designated as the “normal” APP. A 751-amino acid polypeptide (APP751) has been described by Ponte et al. (1988) Nature 331: 525-527 and Tanzi et al. (1988) Nature 331: 528-530. A 770-amino acid isotype of APP (APP770) is described in Kitaguchi et al. (1988) Nature 331: 530-532. A number of specific variants of APP have also been described having mutations which can differ in both position and phenotype. A general review of such mutations is pivoted in Hardy (1992) Nature Genet. 1: 233-235. A mutation of particular interest is designated the “Swedish” mutation where the normal Lys-Met residues at positions 595 and 596 are replaced by Asn-Leu. This mutation is located directly upstream of the normal p-secretase cleavage site of APP, which occurs between residues 596 and 597 of the 695 isotype.

APP is post-translationally processed by several proteolytic pathways resulting in the secretion of various fragments or intracellular fragmentation and degradation. F. Checker, J. Neurochem. 65: 1431-1444 (1995). The combined activity of β-secretase and γ-secretase on APP releases an intact β-amyloid peptide (Aβ), which is a major constituent of amyloid plaques. Initial cleavage of APP by β-secretase generates soluble APP and membrane-associated β-CTF that can be further processed by γ-secretase to generate a 40 or a 42 amino acid peptide (Aβ1-40 or Aβ1-42). Alternatively, APP processing by α-secretase leads to the formation of soluble APP and membrane associated α-CTF the latter being a substrate for γ-secretase to generate the non-amyloidogenic p3. Aβ is an approximately 43 amino acid peptide, which comprises residues 597-640 of the 695 amino acid isotype of APP. Internal cleavage of Aβ by a α-secretase inhibits the release of the full-length Aβ peptide. Although the extent of pathogenic involvement of the secretases in AD progression is not fully elucidated, these proteolytic events are known to either promote or inhibit Aβ formation, and thus are thought to be good therapeutic candidates for AD.

The polytopic transmembrane protein presenilin has been strongly implicated in γ-secretase activity (for review see Haass and De Strooper, Science 286: 916-919 (1999)). Mutagenesis of two transmembrane aspartates of presenilin led to the inactivation of γ-secretase activity in cellular assays (Wolfe et al., Nature 398: 513-517 (1999)).

As a result, both α- and β-CTFs accumulated and Aβ formation was significantly decreased. Similar effects were seen upon inhibition of γ-secretase using substrate analogs (Wolfe et al., J. Med. Chem. 41: 6-9 (1998)). While it remains to be determined whether presenilin is sufficient as γ-secretase or whether it requires another unique co-factor of so far unknown nature to exert its function Presenilin 1 and γ-secretase activity have recently been shown to co-precipitate from membrane extracts (Li et al. Proc. Natl. Acad. Sci. USA 97 (11): 6138-43 (2000)).

As discussed above, there are at least two proteases involved in the generation of Aβ, referred to as β- and γ-secretases (Citron et al., Neuron 17: 171-179 (1996); Seubert et al., Nature 361: 260-263 (1993); Cai et al., Science 259: 514-516 (1993); and Citron et al., Neuron 14: 661-670 (1995)). There have been intense efforts in recent years to identify and characterize these enzymes. Recently five independent groups have reported cloning and characterization of genes corresponding to a β-secretase (Vassar et ah, Science 286: 735-741 (1999); Yan et al., Nature 402: 533-537 (1999); Sinha et al., Nature 402: 537-540 (1999); Hussain et al., Mol. Cell. Neurosci. 14: 419-427 (1999); Lin et al. Proc. Natl. Acad. Sci. USA 97: 1456-1460 (2000)). The membrane-bound aspartyl protease has been variously referred to as P-site APP-cleaving enzyme (BACE), Aspartyl protease-2 (Asp2), memapsin 2 or simply as β-secretase. However, the deduced amino acid sequence of the polypeptide chain reported by all five groups is identical. The cloned enzyme possesses many of the characteristics expected of an authentic β-secretase. In particular, BACE overexpression resulted in an increase in both β-NTF and Aβ levels while suppression of BACE with antisense oligonucleotides led to a significant reduction of these cleavage products. As predicted for the genuine β-secretase, the Swedish double mutant of APP (APPsw, Mullan et al., Nature Genetics 1: 345-347 (1992); Citron et al, Nature 360: 672-674 (1992); Cai et al., Science 259: 514-516 (1993)) was cleaved more efficiently by BACE. Taken together, these results have led to the notion that BACE is the main β-secretase activity.

A close homolog of BACE, designated DRAP or BACE2, has been described (Acquati et al., FEBS Lett. 468: 59-64 (2000), GenBank accession numbers for the human and mouse cDNA sequences: AF050171 and AF051150, respectively; Bennett et al., J. Biol. Chem. 275: 37712-7 (2000)). BACE and BACE2 share 64% amino acid similarity but the role of BACE2 in APP processing has not yet been elucidated. Strikingly, BACE2 expression in brain appears to be very low and this observation has contributed to the assumption that BACE2's role in β-secretase cleavage might only be minor (Bennett et al., ibid).

Meprins are zinc-dependent, membrane-bound proteases and members of the “astacin family” of metalloproteinases (Bond and Beynon, Protein Sci. 4: 1247-1261 (1995)). The enzymes are multidomain, oligomeric proteins. The expression is highly regulated on the transcriptional and translational level. Typically, the proteins are targeted to apical membranes of polarized epithelial cells (Eldering et al., Eur. J. Biochem. 247: 920-932 (1997)). Meprins have been identified in leukocytes, cancer cells and intestine and kidney.

Meprins consist of two types of subunits, α and β, that are encoded by two genes: Mep1a on human chromosome 6p21.2-p21.1 and Mep1b on human chromosome 18q12.2-q12.3) (Bond et al., Genomics 25: 300-303 (1995)).

Meprins have been implicated in kidney fibrosis, injury, and end-stage kidney disease (Ricardo et al., Am. J. Physiol. 270:F669-676 (1996); Sampson et al., J. Biol. Chem. 276: 34128-34188 (2001); Trachtmann et al., Biochem. Biophys. Res. Commun. 208: 498-505 (1995)).

The amino acid sequences of meprin α and β are 42% identical and the domain structures are similar (Jiang et al., J. Biol. Chem. 267: 9185-9193 (1992)). Meprin-α and -β form homo- or heterodimers. Due to an insertion of a domain in meprin-α, it can undergo cleavage and might be secreted, whereas meprin-β remains an integral type 1 transmembrane protein. Homooligomers of meprin-β (meprin B) are primarily membrane-bound dimers. Meprin A is any isoform containing the meprin-α subunit.

Meprin-α and -β have distinct peptide bond specificities. Meprin-β prefers peptides containing acidic amino acids near the scissile bond, meprin-α prefers small or hydrophobic amino acids at the cleavage site.

However, no pathway or enzyme is known so far that is able to release N-terminal truncated forms of Aβ, such as Aβ3-40, Aβ3-42, Aβ11-40 and/or Aβ11-42, from APP.

### SUMMARY OF THE INVENTION

Experimental data disclosed herein describe for the first time that the endopeptidases meprin-α and meprin-β possess-secretase activity when reconstituting β-secretase cleavage in a cell-free assay using wild-type (wt) or Swedish mutant forms of APP751 as a substrate. The invention is further based on the unexpected finding that meprin-α as well as meprin-β cleave APP at various cleavage sites within or before the sequence of Aβ. Moreover, this invention describes for the first time proteases that are able to cleave APP after the alanine residue at position number 2 at the N-terminus of the Aβ peptide sequence, which produces a free glutamic acid residue at position number 3 at the N-terminus of the Aβ peptide sequence. Together with the action of γ-secretase, there is identified herein for the first time a pathway that is able to release N-terminally truncated forms of Aβ, such as Aβ3-x, i.e. Aβ3-40, and Aβ3-42, from APP.

The present invention further provides antagonists of meprin-α and/or meprin-β, compositions comprising said meprin-α and/or meprin-β antagonists, and the use of said antagonists or compositions in the treatment of Amyloidosis.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 shows the MALDI-TOF spectra of substrate wt (H-GLTNIKTEEISEVKMDAEFRHDSGYEVHHQ-NH₂) before (0 h) and after cleavage with the subunit meprin-α (2 h). The fragments 5-30, 7-30, 17-30 and 16-30 were identified.

FIG. 2 shows the MALDI-TOF spectra of substrate wt (H-GLTNIKTEEISEVKMDAEFRHDSGYEVHHQ-NH₂) before (0 h) and after cleavage with the subunit meprin-β (2 h). The fragments 1-16, 1-15, 17-30, and 18-30 were identified.

FIG. 3 shows the MALDI-TOF spectra of substrate D597isoD (H-GLTNIKTEEISEVKMiDAEFRHDSGYEVHHQ-NH₂) before (0 h) and after cleavage with the subunit meprin-α (2 h). The fragments 5-30, 7-30, 1-21, 1-15, 17-30 and 16-30 were identified.

FIG. 4 shows the MALDI-TOF spectra of substrate D597isoD (H-GLTNIKTEEISEVKMiDAEFRHDSGYEVHHQ-NH₂) before (0 h) and after cleavage with the subunit meprin-β (2 h). The fragments 1-15, 1-16, 17-30 and 16-30 were identified.

FIG. 5 shows the MALDI-TOF spectra of substrate E599Q (H-GLTNIKTEEISEVKMDAQFRHDSGYEVHHQ-NH₂) before (0 h) and after cleavage with the subunit meprin-α (2 h). The fragments 5-30, 7-30, 5-21, 12-30 and 7-21 were identified.

FIG. 6 shows the MALDI-TOF spectra of substrate E599Q (H-GLTNIKTEEISEVKMDAQFRHDSGYEVHHQ-NH₂) before (0 h) and after cleavage with the subunit meprin-β (2 h). The fragments 8-30, 9-30, 1-22, 1-21, 12-30 17-30 18-30 9-21 were identified.

FIG. 7 shows the MALDI-TOF spectra of substrate sw (H-GLTNIKTEEISEVNLDAEFRHDSGYEVHHQ-NH₂) before (0 h) and after cleavage with the subunit meprin-α (2 h). The fragments 5-30, 7-30 and 16-30 were identified.

FIG. 8 shows the MALDI-TOF spectra of substrate sw (H-GLTNIKTEEISEVNLDAEFRHDSGYEVHHQ-NH₂) before (0 h) and after cleavage with the subunit meprin-β (2 h). The fragments 16-30, 17-30 1-15 and 18-30 were identified.

FIG. 9 shows the MALDI-TOF spectra of substrate sw D597isoD (H-GLTNIKTEEISEVNLiDAEFRHDSGYEVHHQ-NH₂) before (0 h) and after cleavage with the subunit meprin-α (2 h). The fragments 5-30 and 7-30 were identified.

FIG. 10 shows the MALDI-TOF spectra of substrate sw D597isoD (H-GLTNIKTEEISEVNLiDAEFRHDSGYEVHHQ-NH₂) before (0 h) and after cleavage with the subunit meprin-β (2 h). The fragment 12-30 were identified.

FIG. 11 shows the MALDI-TOF spectra of substrate sw E599Q (H-GLTNIKTEEISEVNLDAQFRHDSGYEVHHQ-NH₂) before (0 h) and after cleavage with the subunit meprin-α (2 h). The fragments 1-21, 7-30 and 5-30 were identified.

FIG. 12 shows the MALDI-TOF spectra of substrate sw E599Q (H-GLTNIKTEEISEVNLDAQFRHDSGYEVHHQ-NH₂) before (0 h) and after cleavage with the subunit meprin-β (2 h). The fragments 16-30, 1-15, 9-21 and 9-20 were identified.

FIG. 13 shows the schematic illustration of identified cleavage sites of the subunit meprin-α investigated in substrates bearing β-secretase cleavage site.

FIG. 14 shows the schematic illustration of identified cleavage sites of the subunit meprin-β investigated in substrates bearing β-secretase cleavage site.

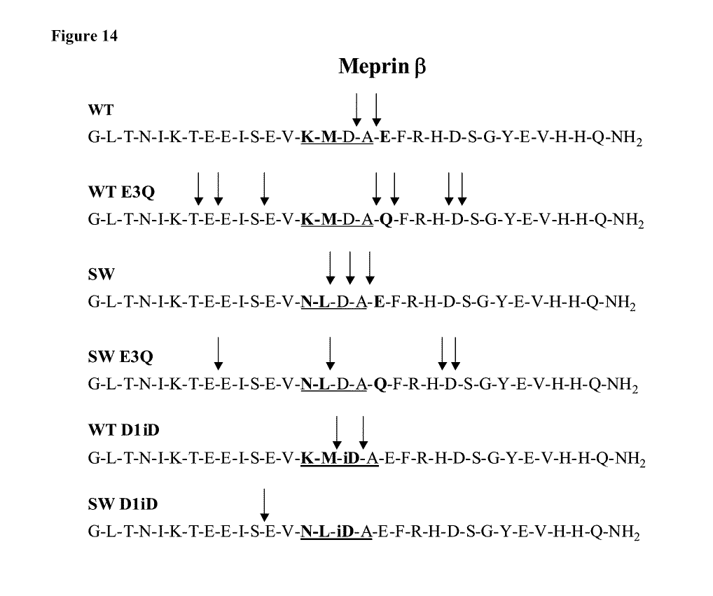

FIG. 15 shows the secretion of Aβx-40 in HEK293 cells after stable transfection with pIRES-hAPP.

FIG. 16 shows the amino acid sequence of human meprin-α (SEQ ID NO: β).

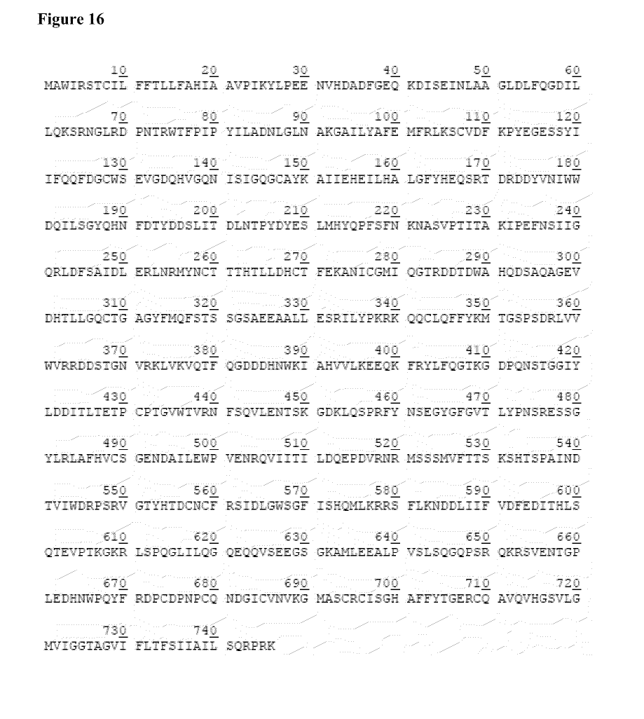

FIG. 17 shows the amino acid sequence of murine meprin-α (SEQ ID NO: 14).

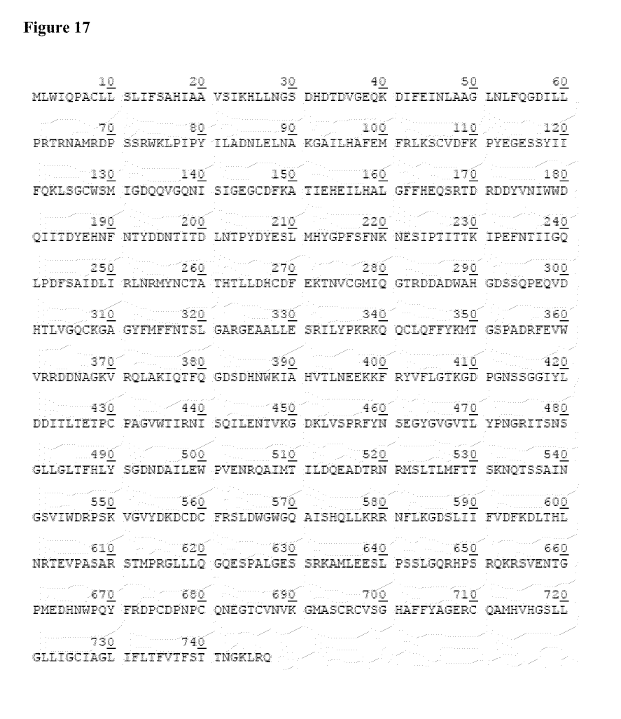

FIG. 18 shows the amino acid sequence of human meprin-β (SEQ ID NO: 15).

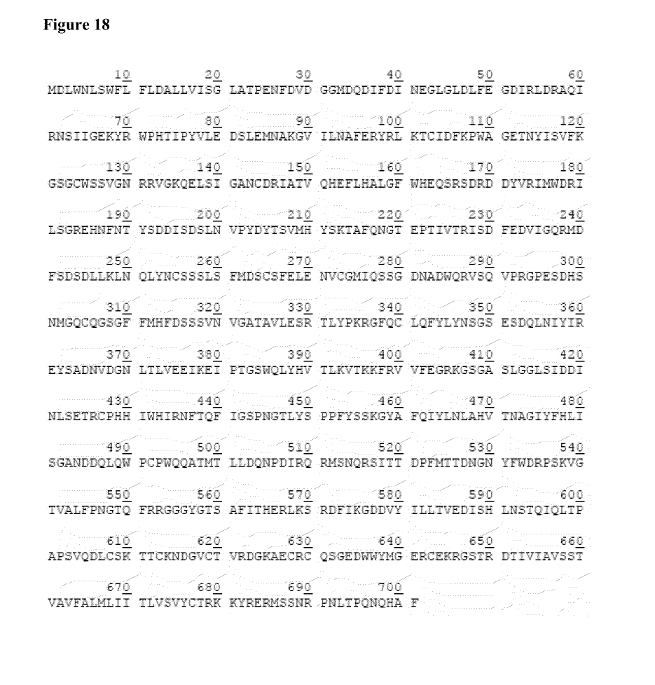

FIG. 19 shows the amino acid sequence of murine meprin-β (SEQ ID NO: 16).

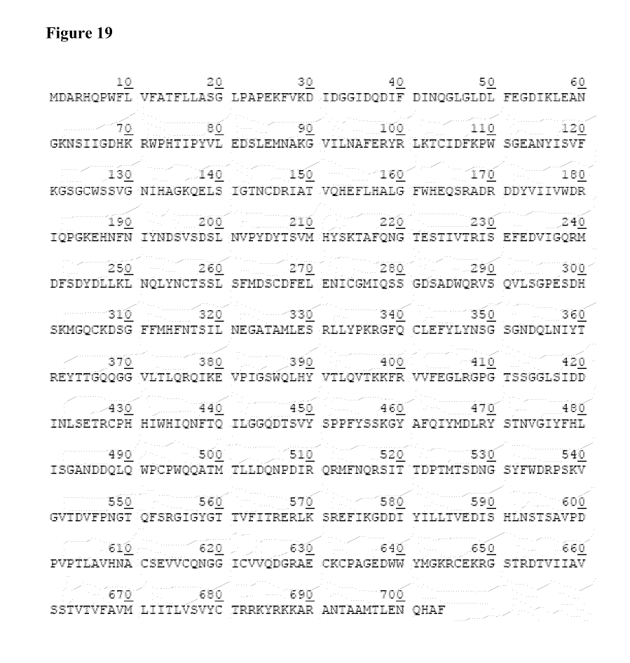

FIG. 20 shows the amino acid sequence of human APP isoform 695 (SEQ ID NO: 17).

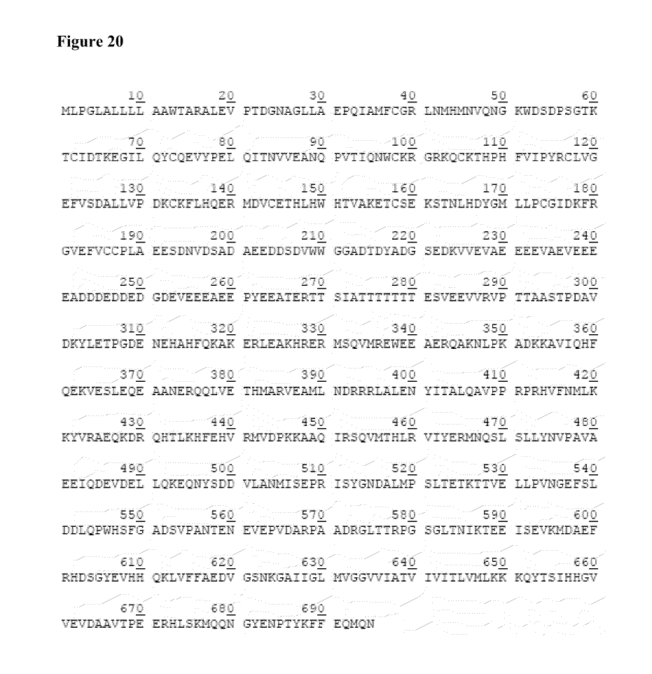

FIG. 21 shows the amino acid sequence of murine APP isoform 695 (SEQ ID NO: 18).

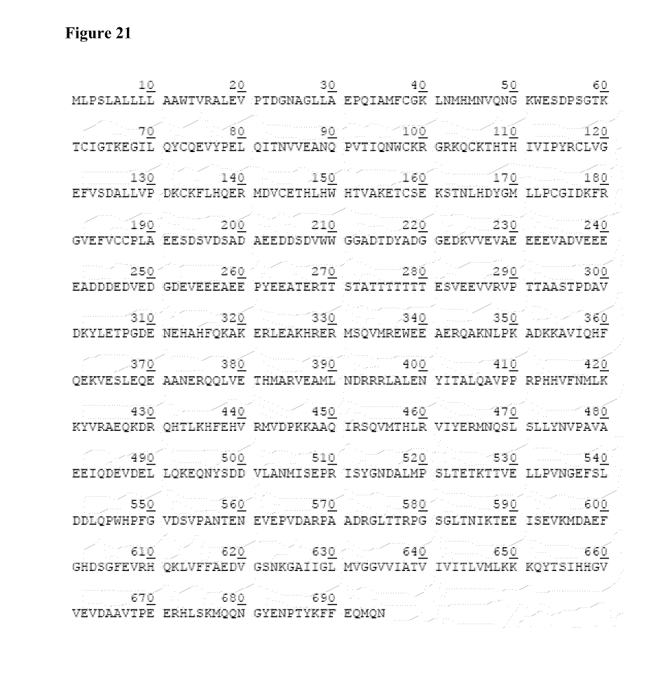

FIG. 22 shows Aβ secreted by HEK293 cells after transient transfection with human meprin-β (hMPβ) (** for P<0.01). Secretion of Aβ produced from endogenous APP is increased after transfection with pcDNA-hMPβ.

FIG. 23 shows Aβ secreted by HEK293 cells after transient transfection with human meprin-β (hMPβ) and human APP (** for P<0.01, *** for P<0.001). Secretion of Aβ is significantly increased after transfection with pcDNA-hMPβ and pcDNA-hAPP wildtyp and E3Q.

FIG. 24 shows Aβ secreted by HEK293 cells after transient transfection with human meprin-β (hMPβ) and human APP estimated with antibody 6E10(** for P<0.01, *** for P<0.001). Secretion of Aβ is significantly increased after transfection with pcDNA-hMPβ and pcDNA-hAPP wildtyp, swedish and E3Q. The increase is due to the formation of Aβ starting at least at N-terminal position 7 (Aβ8-40/42 is not detected by 6E10).

FIG. 25 shows Aβ secreted by HEK293 cells after transient transfection with human meprin-β (hMPβ) and human APP—urea western blot after immuno-precipitation with standard peptides

FIG. 26 shows Aβ secreted by HEK293 cells after transient transfection with human meprin-β (hMPβ) and human APP—Influence of the matrix-metalloprotease inhibitor actinonin (20 μM).

### DETAILED DESCRIPTION OF THE INVENTION

### Definitions

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

As used herein, “β-amyloid precursor protein” (APP or β-APP) refers to a polypeptide that is encoded by a gene of the same name localized in humans on the long arm of chromosome 21 and that includes a β-amyloid protein region within its carboxy terminal region.

The term “meprin-α” is used herein to refer to a native sequence of meprin-α from any animal, e.g. mammalian, species, including humans, and meprin-α variants (which are further defined below). The meprin-α polypeptides may be isolated from a variety of sources, including human tissue types or prepared by recombinant and/or synthetic methods.

“Native sequence meprin-α” refers to a polypeptide having the same amino acid sequence as a meprin-α polypeptide occurring in nature regardless of its mode of preparation. A native sequence meprin-α may be isolated from nature, or prepared by recombinant and/or synthetic methods. The term “native sequence meprin-α” specifically encompasses naturally occurring truncated or secreted forms, naturally occurring variant forms (e.g. alternatively spliced forms), and naturally occurring allelic variants of meprin-α, whether known or to be discovered in the future.

The term “meprin-α variant” refers to amino acid sequence variants of a native sequence meprin-α, containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence of meprin-α.

The term “meprin-β” is used herein to refer to a native sequence of meprin-β from any animal, e.g. mammalian, species, including humans, and meprin-β variants (which are further defined below). The meprin-β polypeptides may be isolated from a variety of sources, including human tissue types or prepared by recombinant and/or synthetic methods.

“Native sequence meprin-β” refers to a polypeptide having the same amino acid sequence as a meprin-β polypeptide occurring in nature regardless of its mode of preparation. A native sequence meprin-β may be isolated from nature, or prepared by recombinant and/or synthetic methods. The term “native sequence meprin-β” specifically encompasses naturally occurring truncated or secreted forms, naturally occurring variant forms (e.g. alternatively spliced forms), and naturally occurring allelic variants of meprin-β, whether known or to be discovered in the future.

The term “meprin-β variant” refers to amino acid sequence variants of a native sequence meprin-β, containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence of meprin-β.

“Sequence identity” is defined as the percentage of amino acid residues in a candidate sequence that are identical with the amino acid residues in a native sequence polypeptide after aligning the sequences and introducing gaps, if necessary, to achieve the maximum percent sequence identity, and not considering any conservative substitutions as part of sequence identity. The % sequence identity values are generated by NCBI BLAST2.0 software as defined by Altschul et al. (1997), Nucleic Acids Res. 25: 3389-3402.

The term “recombinant” when used with reference to a cell, animal, or virus indicates that the cell, animal, or virus encodes a foreign DNA or RNA. For example, recombinant cells optionally express nucleic acids (e.g., RNA) not found within the native (non-recombinant) form of the cell.

“Mammal” for purposes of the present invention refers to any animal classified as a mammal, including humans, domestic and farm animals, and zoo, sports, or pet animals, such as dogs, horses, cats, cows, etc.

Preferably, the mammal herein is human.

The term “biological activity” in connection with meprin-α or meprin-β is used to refer to the ability of a meprin-α or meprin-β molecule (including variants of native sequence meprin-α or meprin-β) to modulate the enzymatic production of β-amyloid peptide (Aβ) from the β-amyloid precursor protein (APP) or a fragment thereof. In a preferred embodiment, the meprin-α or meprin-β “biological activity” is the ability to cleave native sequence APP or a fragment thereof or mutant forms thereof.

The term “antagonist of meprin-α or meprin-β” or “antagonist of meprin-α or meprin-β activity” is used in the broadest sense and includes any molecule that partially or fully blocks, inhibits or neutralizes a biological activity of a meprin-α or meprin-β polypeptide.

The terms “polypeptide”, “peptide”, and “protein”, as used herein, are interchangeable and are defined to mean a biomolecule composed of amino acids linked by a peptide bond.

The terms “a”, “an” and “the” as used herein are defined to mean “one or more” and include the plural unless the context is inappropriate.

The language “diseases and disorders which are caused by or associated with amyloid or amyloid-like proteins” includes, but is not limited to, diseases and disorders caused by the presence or activity of amyloid-like proteins in monomeric, fibril, or polymeric state, or any combination of the three. Such diseases and disorders include, but are not limited to, amyloidosis, endocrine tumors, and macular degeneration.

The term “amyloidosis” refers to a group of diseases and disorders associated with amyloid plaque formation including, but not limited to, primary amyloidosis, secondary amyloidosis and age-related amyloidosis such as diseases including, but not limited to, neurological disorders such as Alzheimer's Disease (AD), including diseases or conditions characterized by a loss of cognitive memory capacity such as, for example, mild cognitive impairment (MCI), sporadic Alzheimer's disease, Lewy body dementia, Down's syndrome, hereditary cerebral hemorrhage with amyloidosis (Dutch type), non-traumatic cerebral hemorrhage of the elderly; the Guam Parkinson-Dementia complex, familial forms of Alzheimer's disease, like Familial British Dementia (FBD), Familial Danish Dementia (FDD), familial polyneuropathy (Iowa), familial amyloidosis (Finnish) and hereditary cerebral hemorrhage (Icelandic); as well as other diseases which are based on or associated with amyloid-like proteins such as progressive supranuclear palsy, multiple sclerosis; bovine spongiform encephalopathy (BSE), Creutzfeld Jacob disease, scrapie, Parkinson's disease, HIV-related dementia, ALS (amyotropic lateral sclerosis), inclusion-body myositis (IBM), Adult Onset Diabetes, and senile cardiac amyloidosis; and various eye diseases including macular degeneration, drusen-related optic neuropathy, and cataract due to beta-amyloid deposition; chronic inflammatory conditions (e.g., tuberculosis, osteomyelitis, and the like); non-infectious conditions such as juvenile rheumatoid arthritis, ankylosing spondylitis and Crohn's disease and the like; medullary carcinoma of the thyroid, atrial amyloid, and diabetes mellitus (insulinomas). Further examples of diseases included within the definition of amyloidosis may be found in Louis W. Heck, “The Amyloid Diseases” in Cecil's Textbook of Medicine 1504-6 (W. B. Saunders & Co., Philadelphia, Pa.; 1996).

“Amyloid β, Aβ or β-amyloid” is an art recognized term and refers to amyloid β proteins and peptides, amyloid β precursor protein (APP), as well as modifications, fragments and any functional equivalents thereof. In particular, by amyloid β as used herein is meant any fragment produced by proteolytic cleavage of APP but especially those fragments which are involved in or associated with the amyloid pathologies including, but not limited to, Aβ₁₋₃₈, Aβ₁₋₄₀, Aβ₁₋₄₂. The amino acid sequences of these Aβ peptides are as follows:

Aβ 1-42 (SEQ ID NO. 1):
Asp-Ala-Glu-Phe-Arg-His-Asp-Ser-Gly-Tyr-Glu-Val-
His-His-Gln-Lys-Leu-Val-Phe-Phe-Ala-Glu-Asp-Val-
Gly-Ser-Asn-Lys-Gly-Ala-Ile-Ile-Gly-Leu-Met-Val-
Gly-Gly-Val-Val-Ile-Ala
Aβ 1-40 (SEQ ID NO. 2):
Asp-Ala-Glu-Phe-Arg-His-Asp-Ser-Gly-Tyr-Glu-Val-
His-His-Gln-Lys-Leu-Val-Phe-Phe-Ala-Glu-Asp-Val-
Gly-Ser-Asn-Lys-Gly-Ala-Ile-Ile-Gly-Leu-Met-Val-
Gly-Gly-Val-Val
Aβ 1-38 (SEQ ID NO. 3):
Asp-Ala-Glu-Phe-Arg-His-Asp-Ser-Gly-Tyr-Glu-Val-
His-His-Gln-Lys-Leu-Val-Phe-Phe-Ala-Glu-Asp-Val-
Gly-Ser-Asn-Lys-Gly-Ala-Ile-Ile-Gly-Leu-Met-Val-
Gly-Gly

“pGlu-Aβ” or “Aβ N3pE” refers to N-terminally truncated forms of Aβ, that start at the glutamic acid residue at position 3 in the amino acid sequence of Aβ, and wherein said glutamic acid residue is cyclized to form a pyroglutamic acid residue. In particular, by pGlu-Aβ as used herein are meant those fragments which are involved in or associated with the amyloid pathologies including, but not limited to, pGlu-Aβ₃₋₃₈, pGlu-Aβ₃₋₄₀, p-Glu-Aβ₃₋₄₂.

The sequences of the N-terminally truncated forms of Aβ, Aβ₃₋₃₈, Aβ₃₋₄₀, Aβ₃₋₄₂ are as follows:

Aβ 3-42 (SEQ ID NO. 4):
Glu-Phe-Arg-His-Asp-Ser-Gly-Tyr-Glu-Val-His-His-
Gln-Lys-Leu-Val-Phe-Phe-Ala-Glu-Asp-Val-Gly-Ser-
Asn-Lys-Gly-Ala-Ile-Ile-Gly-Leu-Met-Val-Gly-Gly-
Val-Val-Ile-Ala
Aβ 3-40 (SEQ ID NO. 5):
Glu-Phe-Arg-His-Asp-Ser-Gly-Tyr-Glu-Val-His-His-
Gln-Lys-Leu-Val-Phe-Phe-Ala-Glu-Asp-Val-Gly-Ser-
Asn-Lys-Gly-Ala-Ile-Ile-Gly-Leu-Met-Val-Gly-Gly-
Val-Val
Aβ 3-38 (SEQ ID NO. 6):
Glu-Phe-Arg-His-Asp-Ser-Gly-Tyr-Glu-Val-His-His-
Gln-Lys-Leu-Val-Phe-Phe-Ala-Glu-Asp-Val-Gly-Ser-
Asn-Lys-Gly-Ala-Ile-Ile-Gly-Leu-Met-Val-Gly-Gly

The terms “APP secretase”, “secretase” and “secretase activity” as used interchangeably herein refer to any proteolytic enzyme and/or activity which results in the secretion of various fragments or intracellular fragmentation and degradation of APP. This includes α-secretase, β-secretase, γ-secretase, and any similar but as of yet unidentified enzymes, which cause the proteolysis of either APP or Aβ.

The terms “β-secretase” and “β-secretase activity” as used interchangeably herein refer to the enzyme or enzymes responsible for proteolysis of APP at the N-terminal cleavage site of APP, which occurs between residues 596 and 597 of the 695 isotype of APP (Kang et al. (1987) Nature 325: 733-736) and between residues 652 and 653 of the 751 isotype of APP (Ponte et al. (1988) Nature 331: 525-527). A secondary cleavage by β-secretase occurs between residues 605 and 606 of the 695 APP isoform and between residues 661 and 662 of the 751 APP isoform (Higaki et al. (1996) Neuron 14: 651-659). The terms are used in the broadest sense and include isolated, partially or fully purified, recombinantly produced enzymes, cells or cell preparations (including membrane preparations) comprising a β-secretase enzyme, and any solution or mixture comprising a β-secretase enzyme.

The term “α-secretase” and “α-secretase activity” are used interchangeably, and refer to the enzyme or enzymes capable of producing a cleavage within the β-amyloid domain of APP or the C-terminal fragment of APP resulting from p-secretase processing. The processing by α-secretase activity, generally occurs between residues 612 and 613 of the 695 APP isoform or between residues 16 and 17 of the C-terminal fragment of APP resulting from 3-secretase processing. The terms are used in the broadest sense and include isolated, partially or fully purified, recombinantly produced enzymes, cells or cell preparations (including membrane preparations) comprising an α-secretase enzyme, and any solution or mixture comprising a α-secretase enzyme.

The terms “γ-secretase” and “γ-secretase activity” are used interchangeably, and refer to the enzyme or enzymes responsible for generating the C-termini of the β-amyloid peptides by cleaving within the transmembrane region of APP. The terms are used in the broadest sense and include isolated, partially or fully purified, recombinantly produced enzymes, cells or cell preparations (including membrane preparations) comprising an γ-secretase enzyme, and any solution or mixture comprising a γ-secretase enzyme.

The term “Alzheimer's disease” (abbreviated herein as “AD”) as used herein refers to a condition associated with formation of neuritic plaques comprising β-amyloid protein primarily in the hippocampus and cerebral cortex, as well as impairment in both learning and memory. “AD” as used herein is meant to encompass both AD as well as AD-type pathologies.

The term “AD-type pathology” as used herein refers to a combination of CNS alterations including, but not limited to, formation of neuritic plaques containing β-amyloid protein in the hippocampus and cerebral cortex. Such AD-type pathologies can include, but are not necessarily limited to, disorders associated with aberrant expression and/or deposition of APP, overexpression of APP, expression of aberrant APP gene products, and other phenomena associated with AD. Exemplary AD-type pathologies include, but are not necessarily limited to, AD-type pathologies associated with Down's syndrome that are associated with overexpression of APP.

The term “phenomenon associated with Alzheimer's disease” as used herein refers to a structural, molecular, or functional event associated with AD, particularly such an event that is readily assessable in an animal model. Such events include, but are not limited to, amyloid deposition, neuropathological developments, learning and memory deficits, and other AD-associated characteristics.

The term “cerebral amyloid angiopathy” (abbreviated herein as CAA) as used herein refers to a condition associated with formation of amyloid deposition within cerebral vessels which can be complicated by cerebral parenchymal hemorrhage. CAA is also associated with increased risk of stroke as well as development of cerebella and subarachnoid hemorrhages (Vinters (1987) Stroke 18: 311-324; Haan et al. (1994) Dementia 5: 210-213; Itoh, et al. (1993) J. Neurol. Sci. 116: 135-414). CAA can also be associated with dementia prior to onset of hemorrhages.

The vascular amyloid deposits associated with CAA can exist in the absence of AD, but are more frequently associated with AD.

The term “phenomenon associated with cerebral amyloid angiopathy” as used herein refers to a molecular, structural, or functional event associated with CAA, particularly such an event that is readily assessable in an animal model. Such events include, but are not limited to, amyloid deposition, cerebral parenchymal hemorrhage, and other CAA-associated characteristics.

The term “β-amyloid deposit” as used herein refers to a deposit in the brain composed of Aβ as well as other substances.

The term “non-amyloidogenic” refers to a process which reduces or eliminates the production of β-amyloid.

The term “compound” as used herein describes any molecule, e.g., protein, naturally occurring substances, synthesized protein or small molecule pharmaceutical, with the capability of affecting secretase activity. Such compounds may be used to treat the molecular and clinical phenomena associated with amyloid-associated disorders, and specifically AD, CAA and prion-medicated disorder.

The terms “effective dose”, “effective amount” and “amount effective” are used interchangeably, and refer to an administration of a compound sufficient to provide the desired physiological and/or psychological change. This will vary depending on the patient, the disease and the treatment. The dose may either be a therapeutic dose, in which case it should sufficiently alter levels of amyloid plaques in the subject to alleviate or ameliorate the symptoms of the disorder or condition, or a prophylactic dose, which should be sufficient to prevent accumulation of amyloid plaques to an undesirable level.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease.

The therapeutic agents that can be identified using the assay of the invention are particularly useful in the treatment of any disease associated with the deposition of β-amyloid, including AD, hereditary cerebral hemorrhage with amyloidosis, and prion-mediated disorders, and the like.

The terms “modulate”, “alter” and grammatical variants thereof, when used in connection with the methods of the present invention, include any and all modifications, such as inhibition or enhancement of β-secretase activity.

Inhibitors, in particular inhibitors of meprin-α and meprin-β.

Reversible enzyme inhibitors: comprise competitive inhibitors, non-competitive reversible inhibitors, slow-binding or tight-binding inhibitors, transition state analogues and multisubstrate analogues.

### Competitive Inhibitors Show

i) non-covalent interactions with the enzyme,
ii) compete with substrate for the enzyme active site.

The principal mechanism of action of a reversible enzyme inhibitor and the definition of the dissociation constant can be visualized as follows:

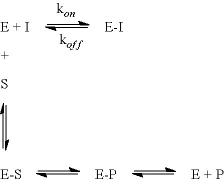

$${K}_{D} = {K}_{i} = \frac{{k}_{off}}{{k}_{on}}$$

The formation of the enzyme-inhibitor [E-I] complex prevents binding of substrates, therefore the reaction cannot proceed to the normal physiological product, P. A larger inhibitor concentration [I] leads to larger [E-I], leaving less free enzyme to which the substrate can bind.

### Non-Competitive Reversible Inhibitors

i) bind at a site other than active site (allosteric binding site)
ii) cause a conformational change in the enzyme which decreases or stops catalytic activity.

### Slow-Binding or Tight-Binding Inhibitors

i) are competitive inhibitors where the equilibrium between inhibitor and enzyme is reached slowly,
ii) (kₒₙ is slow), possibly due to conformational changes that must occur in the enzyme or inhibitor
a) are often transition state analogues
b) are effective at concentrations similar to the enzyme concentration (subnanomolar KD values)
c) due to k_{off} values being so low these types of inhibitors are “almost” irreversible.

### Transition State Analogues

Are competitive inhibitors which mimic the transition state of an enzyme catalyzed reaction. Enzyme catalysis occurs due to a lowering of the energy of the transition state, therefore, transition state binding is favored over substrate binding.

### Multisubstrate Analogues

For a reaction involving two or more substrates, a competitive inhibitor or transition state analogue can be designed which contains structural characteristics resembling two or more of the substrates.

Irreversible enzyme inhibitors: drive the equilibrium between the unbound enzyme and inhibitor and enzyme inhibitor complex (E+I< - - - > E-I) all the way to the E-1-side with a covalent bond (˜100 kcal/mole), making the inhibition irreversible.

### Affinity Labeling Agents

Active-site directed irreversible inhibitors (competitive irreversible inhibitor) are recognized by the enzyme (reversible, specific binding) followed by covalent bond formation, and
i) are structurally similar to substrate, transition state or product allowing for specific interaction between drug and target enzyme,
ii) contain reactive functional group (e.g. a nucleophile, —COCH₂Br) allowing for covalent bond formation.

The reaction scheme below describes an active-site directed reagent with its target enzyme where K_{D} is the dissociation constant and k_{inactivation} is the rate of covalent bond formation.

$$E + I {}^{{K}_{D}} E \cdot I {}^{{k}_{inactivation}} E - I$$

Mechanism-based enzyme inactivators (also called suicide inhibitors) are active-site directed reagents (unreactive) which bind to the enzyme active site where they are transformed to a reactive form (activated) by the enzyme's catalytic capabilities. Once activated, a covalent bond between the inhibitor and the enzyme is formed.

The reaction scheme below shows the mechanism of action of a mechanism based enzyme inactivator, where K_{D} is the dissociation complex, k₂ is the rate of activation of the inhibitor once bound to the enzyme, k₃ is the rate of dissociation of the activated inhibitor, P, from the enzyme (product can still be reactive) from the enzyme and k₄ is the rate of covalent bond formation between the activated inhibitor and the enzyme.

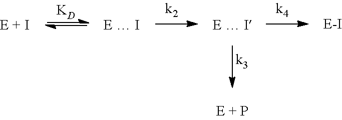

Inactivation (covalent bond formation, k₄) must occur prior to dissociation (k₃) otherwise the now reactive inhibitor is released into the environment. The partition ratio, k₃/k₄: ratio of released product to inactivation should be minimized for efficient inactivation of the system and minimal undesirable side reactions.

A large partition ratio (favors dissociation) leads to nonspecific reactions.

Uncompetitive enzyme inhibitors: As a definition of uncompetitive inhibitor (an inhibitor which binds only to ES complexes) the following equilibria equation can be assumed:

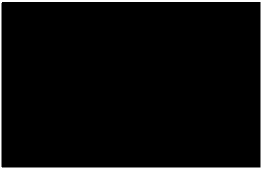

The ES complex dissociates the substrate with a dissociation constant equal to Ks, whereas the ESI complex does not dissociate it (i.e has a Ks value equal to zero). The Km's of Michaelis-Mententype enzymes are expected to be reduced. Increasing substrate concentration leads to increasing ESI concentration (a complex incapable of progressing to reaction products) therefore the inhibition cannot be removed.

Preferred according to the present invention are competitive enzyme inhibitors.

Most preferred are competitive reversible enzyme inhibitors.

The terms “Kᵢ” or “K_{I}” and “K_{D}” are binding constants, which describe the binding of an inhibitor to and the subsequent release from an enzyme. Another measure is the “IC₅₀” value, which reflects the inhibitor concentration, which at a given substrate concentration results in 50% enzyme activity.

### In Particular the Present Invention Pertains to the Following Items:

1. A method of modulating the enzymatic production of β-amyloid peptide (Aβ) from β-amyloid precursor protein (APP) or a fragment thereof, comprising contacting said APP or APP fragment with a meprin-α and/or meprin-β polypeptide or an antagonist thereof.

2. The method of item 1 wherein said APP is a native sequence human APP.

3. The method of item 1 or 2 wherein said APP is the 695-amino acid isotype.

4. The method according to any one of the preceding items, wherein said APP contains the Swedish mutation.

5. The method according to any one of the preceding items, wherein said meprin-α is a native sequence meprin-α polypeptide.

6. The method according to any one of the preceding items, wherein said meprin-β is a native sequence meprin-β polypeptide.

7. A method of inhibiting the formation of a β-amyloid peptide (Aβ) from β-amyloid precursor protein (APP) or a fragment thereof, comprising contacting said APP or APP fragment with an antagonist of meprin-α and/or meprin-β.

8. The method of item 7, wherein said APP is a native sequence human APP.

9. The method of item 7 or 8, wherein said APP is the 695-amino acid isotype.

10. The method according to any one of items 7 to 9, wherein said APP contains the Swedish mutation.

11. The method according to any one of items 7 to 10, wherein said antagonist inhibits the formation of a β-amyloid peptide (Aβ) from β-amyloid precursor protein (APP) or a fragment thereof, which is catalyzed by meprin-α.

12. The method according to any one of items 7 to 10, wherein said antagonist inhibits the formation of a β-amyloid peptide (Aβ) from β-amyloid precursor protein (APP) or a fragment thereof, which is catalyzed by meprin-β.

13. The method according to any one of items 7 to 12, which is performed in the presence of an α-secretase activity.

14. The method according to any one of items 7 to 12, which is performed in the presence of an γ-secretase activity.

15. The method according to any one of items 7 to 12, which is performed in the presence of a β-secretase activity other than meprin-α and/or meprin-β.

16. The method of item 15, which is performed in the presence of BACE1 or BACE2.

17. The method according to any one of items 7 to 16, wherein said meprin-α and/or meprin-β is in isolated form.

18. The method according to any one of items 7 to 16, wherein said meprin-α and/or meprin-β is in immobilized or cell bound form.

19. The method according to any one of items 7 to 18, wherein said APP or APP fragment is contacted with an antagonist of meprin-α.

20. The method according to any one of items 7 to 18, wherein said APP or APP fragment is contacted with an antagonist of meprin-β.

21. The method according to any one of the preceding items, wherein said antagonist of meprin-α and/or meprin-β is an inhibitor.

22. The method according to any one of the preceding items, wherein said antagonist of meprin-α and/or meprin-β is a competitive inhibitor.

23. The method of item 21 or 22, wherein said inhibitor is a small molecule.

24. A method of inhibiting the release of a full-length Aβ polypeptide from APP or a fragment thereof, comprising cleaving said APP or APP fragment by a meprin-α and/or meprin-β polypeptide.

25. A method for identifying a modulator of the enzymatic production of Aβ from APP or a fragment thereof, comprising contacting APP or an APP fragment and meprin-α and/or meprin-β with a candidate compound and monitoring the effect of the candidate compound on the production of Aβ.

26. The method of item 25, wherein said modulator is an inhibitor of Aβ production.

27. The method of item 26, wherein the effect of the candidate compound on the production of Aβ is monitored by measuring the amount of Aβ formed.

28. The method according to any one of items 25 to 27, wherein the effect of the candidate compound on the production of Aβ is monitored by measuring the amount of Aβ1-40 and/or Aβ1-42 formed.

29. The method according to any one of items 25 to 27, wherein the effect of the candidate compound on the production of Aβ is monitored by measuring the amount of Aβ3-40 and/or Aβ3-42 formed.

30. The method according to any one of items 25 to 29, which is performed in the presence of an α-secretase activity.

31. The method according to any one of items 25 to 29, which is performed in the presence of an γ-secretase activity.

32. The method according to any one of items 25 to 29, which is performed in the presence of an β-secretase activity other than meprin-α and/or meprin-β activity.

33. The method of item 32, which is performed in the presence of BACE1 or BACE2.

34. The method according to any one of items 25 to 33, wherein the amount of Aβ formed is reduced by at least about 50%.

35. The method according to any one of items 25 to 33, wherein the amount of Aβ formed is reduced by at least about 75%.

36. The method according to any one of items 25 to 33, wherein the amount of Aβ formed is reduced by at least about 90%.

37. The method according to any one of items 25 to 36, which is performed in a cell-free format.

38. A modulator of the enzymatic production of Aβ from APP or a fragment thereof, identified by the method according to any one of items 25 to 37.

39. The modulator of item 38, which is a meprin-α and/or meprin-β antagonist.

40. The modulator of item 38, which is a meprin-α antagonist.

41. The modulator of item 38, which is a meprin-β antagonist.

42. The modulator according to any one of items 38 to 41, which is an inhibitor.

43. The modulator of item 42, which is a competitive inhibitor.

44. The modulator of item 42 or 43, which is a small molecule.

45. A pharmaceutical composition comprising at least one antagonist of meprin-α and/or meprin-β optionally in combination with one or more pharmaceutically acceptable diluents or carriers.

46. The pharmaceutical composition according to item 45, which comprises additionally at least one compound selected from the group consisting of neutron-transmission enhancers, psychotherapeutic drugs, acetylcholine esterase inhibitors, calcium-channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-D-aspartate glutamate receptor antagonists, non-steroidal anti-inflammatory drugs, antioxidants, and serotonergic receptor antagonists.

47. The pharmaceutical composition according to item 45, which comprises additionally at least one compound, selected from the group consisting of compounds effective against oxidative stress, anti-apoptotic compounds, metal chelators, inhibitors of DNA repair such as pirenzepin and metabolites, 3-amino-1-propanesulfonic acid (3 APS), 1,3-propanedisulfonate (1,3PDS), α-secretase activators, β- and γ-secretase inhibitors, tau proteins, neurotransmitter, β-sheet breakers, attractants for β-amyloid clearing/depleting cellular components, inhibitors of N-terminal truncated amyloid beta including pyroglutamated β-amyloid 3-42, such as inhibitors of glutaminyl cyclase, anti-inflammatory molecules, or cholinesterase inhibitors (ChEIs) such as tacrine, rivastigmine, donepezil, galantamine, niacin and/or memantine, Ml agonists and other drugs including any amyloid or tau modifying drug and nutritive supplements, and nutritive supplements, together with an antibody according to the present invention and, optionally, a pharmaceutically acceptable carrier and/or a diluent and/or an excipient.

48. The pharmaceutical composition according to item 45, which comprises additionally at least one inhibitor of glutaminyl cyclase.

49. A method for reducing the amount of β-amyloid deposits in the central nervous system (CNS) of a mammal comprising administering to said mammal an effective amount of an antagonist of meprin-α and/or meprin-β.

50. An antagonist of meprin-α and/or meprin-β or a pharmaceutical composition according to any one of items 45 to 49 for use in the prevention or treatment of amyloidosis.

51. The antagonist of meprin-α and/or meprin-β or the pharmaceutical composition according to item 50 for use in the prevention or treatment of a disease selected from the group consisting of Kennedy's disease, duodenal cancer with or without Helicobacter pylori infections, colorectal cancer, Zolliger-Ellison syndrome, gastric cancer with or without Helicobacter pylori infections, pathogenic psychotic conditions, schizophrenia, infertility, neoplasia, inflammatory host responses, cancer, malign metastasis, melanoma, psoriasis, impaired humoral and cell-mediated immune responses, leukocyte adhesion and migration processes in the endothelium, impaired food intake, impaired sleep-wakefulness, impaired homeostatic regulation of energy metabolism, impaired autonomic function, impaired hormonal balance or impaired regulation of body fluids, multiple sclerosis, the Guillain-Barré syndrome and chronic inflammatory demyelinizing polyradiculoneuropathy.

52. The antagonist of meprin-α and/or meprin-β or the pharmaceutical composition according to item 51 for use in the prevention or treatment of a disease selected from the group consisting of mild cognitive impairment, Alzheimer's disease, Familial British Dementia, Familial Danish Dementia, neurodegeneration in Down Syndrome and Huntington's disease.

52. A method for the treatment or prevention of amyloidosis comprising administering to a subject in need of such treatment an effective amount of an antagonist of meprin-α and/or meprin-β or a pharmaceutical composition according to any one of items 45 to 49.

53. The method according to item 52 for the prevention or treatment of a disease selected from the group consisting of Kennedy's disease, duodenal cancer with or without Helicobacter pylori infections, colorectal cancer, Zolliger-Ellison syndrome, gastric cancer with or without Helicobacter pylori infections, pathogenic psychotic conditions, schizophrenia, infertility, neoplasia, inflammatory host responses, cancer, malign metastasis, melanoma, psoriasis, impaired humoral and cell-mediated immune responses, leukocyte adhesion and migration processes in the endothelium, impaired food intake, impaired sleep-wakefulness, impaired homeostatic regulation of energy metabolism, impaired autonomic function, impaired hormonal balance or impaired regulation of body fluids, multiple sclerosis, the Guillain-Barré syndrome and chronic inflammatory demyelinizing polyradiculoneuropathy.

54. The method according to item 52 for the prevention or treatment of a disease selected from the group consisting of mild cognitive impairment, Alzheimer's disease, Familial British Dementia, Familial Danish Dementia, neurodegeneration in Down Syndrome and Huntington's disease.

55. Use of an antagonist of meprin-α and/or meprin-β or a pharmaceutical composition according to any one of items 45 to 49 for the production of a medicament for the prevention or treatment of amyloidosis.

56. The use of item 55 for the prevention or treatment of a disease selected from the group consisting of Kennedy's disease, duodenal cancer with or without Helicobacter pylori infections, colorectal cancer, Zolliger-Ellison syndrome, gastric cancer with or without Helicobacter pylori infections, pathogenic psychotic conditions, schizophrenia, infertility, neoplasia, inflammatory host responses, cancer, malign metastasis, melanoma, psoriasis, impaired humoral and cell-mediated immune responses, leukocyte adhesion and migration processes in the endothelium, impaired food intake, impaired sleep-wakefulness, impaired homeostatic regulation of energy metabolism, impaired autonomic function, impaired hormonal balance or impaired regulation of body fluids, multiple sclerosis, the Guillain-Barré syndrome and chronic inflammatory demyelinizing polyradiculoneuropathy.

57. The use of item 56 for the prevention or treatment of a disease selected from the group consisting of mild cognitive impairment, Alzheimer's disease, Familial British Dementia, Familial Danish Dementia, neurodegeneration in Down Syndrome and Huntington's disease.

58. The pharmaceutical composition, antagonist, use or method according to any one of items 45 to 57, wherein said antagonist is a meprin-α antagonist.

59. The pharmaceutical composition, antagonist, use or method according to any one of items 45 to 57, wherein said antagonist is a meprin-β antagonist.

60. The pharmaceutical composition, antagonist, use or method according to any one of items 45 to 59, wherein said antagonist is an inhibitor.

61. The pharmaceutical composition, antagonist, use or method according to item 45 to 60, wherein said antagonist is a competitive inhibitor.

62. The pharmaceutical composition, antagonist, use or method according to any one of items 45 to 61, wherein said antagonist is a small molecule.

63. The composition, method, modulator, composition, antagonist, or use of any one of claims 1-62, wherein the antagonist of meprin-α or meprin-β comprises a compound selected from the group consisting of actinonin, batimastat, galardin, NNGH, PLG-NHOH, Ro 32-7315, TAPI-0, and captopril.

In general, an inhibitor of meprin-α and/or meprin-β identified in an assay described hereinbelow, will reduce the level of β-amyloid plaque in the brain tissue of a mammalian host, including humans. In particular, inhibitors of meprin-α and/or meprin-β will reduce the production of Aβ or an Aβ fragment from β-amyloid precursor protein (APP) or a fragment thereof, including full-length Aβ polypeptides, such as Aβ1-40 or Aβ1-42, and N-terminally truncated forms of Aβ, such as Aβ3-x, i.e. Aβ3-40, and Aβ3-42.

In one aspect, the invention concerns a method of modulating the enzymatic production of β-amyloid peptide (Aβ) from β-amyloid precursor protein (APP) or a fragment thereof comprising contacting said APP or APP fragment with a meprin-α or meprin-β polypeptide and/or an agonist or antagonist thereof.

Suitably, the method concerns the production of Aβ or an Aβ fragment from β-amyloid precursor protein (APP) or a fragment thereof comprising contacting said APP or APP fragment with a meprin-α or meprin-β polypeptide.

More suitably, the method concerns the release of a full-length Aβ polypeptide, such as Aβ1-40 or Aβ1-42, from APP or a fragment thereof, comprising contacting said APP or APP fragment with a meprin-α or meprin-β polypeptide.

Most suitably, the method concerns the production of N-terminally truncated forms of Aβ, such as Aβ3-x, i.e. Aβ3-40, and Aβ3-42, from APP or a fragment thereof comprising contacting said APP or APP fragment with a meprin-α or meprin-β polypeptide.

In another embodiment, the method concerns the inhibition of Aβ production from APP or an APP fragment comprising the use of an antagonist of meprin-α and/or meprin-β.

Suitably, the method concerns the inhibition of the formation of an β-amyloid peptide (Aβ) from β-amyloid precursor protein (APP) or a fragment thereof comprising the use of an inhibitor of meprin-α and/or meprin-β.

More suitably, the method concerns the inhibition of the release of a full-length Aβ polypeptide, such as Aβ1-40 or Aβ1-42, from APP or a fragment thereof, comprising the use of an inhibitor of meprin-α and/or meprin-β.

Most suitably, the method concerns the inhibition of the production of N-terminally truncated forms of Aβ, such as Aβ3-x, e.g. Aβ3-40, and Aβ3-42, from APP or a fragment thereof comprising the use of an inhibitor of meprin-α and/or meprin-β.

Particularly preferred according to the present invention are the aforementioned methods, wherein said methods concern the use of an inhibitor of meprin-β.

Inhibitors of meprin-α and/or meprin-β are known in the art (Kruse, M.-N. et al., Biochem. J. (2004), 378, pp. 383-389, incorporated herein by reference). Structures and Ki-values are shown in Table 1. The methods regarding Ki determination are described in detail in example 6.

TABLE 1
Inhibitory constants of inhibitors of meprin-α and/or meprin-β

| Structure | Name | Kᵢ Meprin α | Kᵢ Meprin β |
|---|---|---|---|
| 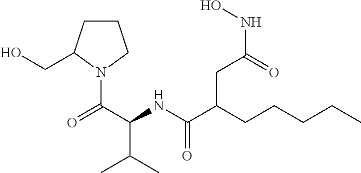 | Actinonin | Kᵢ 2.0 * 10⁻⁸ M ± 2.3 * 10⁻⁹ M | Kᵢ 2.0 * 10⁻⁶ M ± 2.3 * 10⁻⁷ M |
| 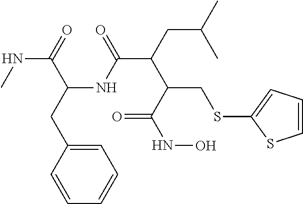 | Batimastat | Kᵢ 4.4 * 10⁻⁶ M ± 4.6 * 10⁻⁷ M | Kᵢ 1.8 * 10⁻⁵ M ± 2.2 * 10⁻⁶ M |
| 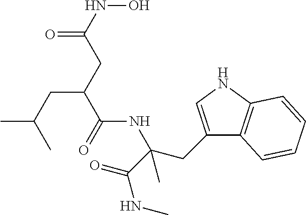 | Galardin | Kᵢ 1.4 * 10⁻⁷ M ± 9.9 * 10⁻⁹ M | Kᵢ 8.9 * 10⁻⁶ M ± 7.1 * 10⁻⁷ M |
| 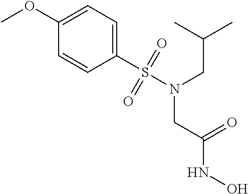 | NNGH | Kᵢ 4.0 * 10⁻⁷ M ± 3.7 * 10⁻⁸ M | Kᵢ 7.4 * 10⁻⁶ M ± 7.6 * 10⁻⁷ M |
| 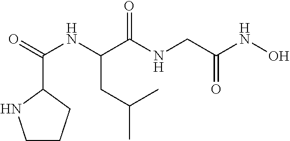 | PLG—NHOH | Kᵢ 5.3 * 10⁻⁷ M ± 5.1 * 10⁻⁸ M | Kᵢ 1.4 * 10⁻⁵ M ± 1.2 * 10⁻⁶ M |
| 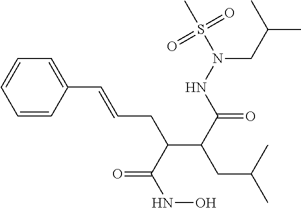 | Ro 32-7315 | Kᵢ 1.6 * 10⁻⁶ M ± 2.6 * 10⁻⁸ M | IC₅₀ 1.6 * 10⁻³ M ± 1.2 * 10⁻⁴ M |
| 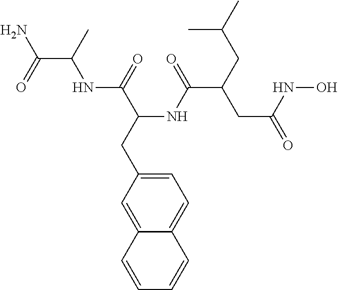 | TAPI-0 | Kᵢ 2.2 * 10⁻⁶ M ± 3.5 * 10⁻⁸ M | IC₅₀ 4.0 * 10⁻⁴ M ± 1.0 * 10⁻⁴ M |
| 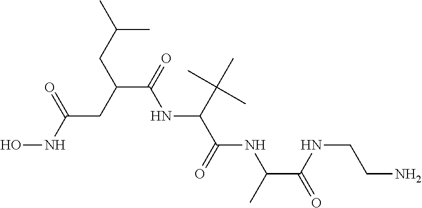 | TAPI-2 | Kᵢ 1.5 * 10⁻⁶ M ± 2.7 * 10⁻⁷ M | IC₅₀ 2.0 * 10⁻⁴ M ± 1.0 * 10⁻⁴ M |
| 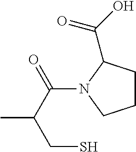 | Captopril | Kᵢ 7.4 * 10⁻⁴ M ± 6.0 * 10⁻⁶ M | Kᵢ 4.1 * 10⁻⁴ M ± 1.2 * 10⁻⁵ M |

Recently, accumulating evidence demonstrates involvement of N-terminally modified Aβ peptide variants in Alzheimer's disease. These N-terminally truncated and pyroGlu modified Aβ N3pE-x peptides, e.g. Aβ N3pE-40 and Aβ N3pE-42, are almost exclusively engrained within plaques of Alzheimer's disease patients. The neurotoxicity of these N-terminally truncated and pyroGlu modified Aβ has been described previously. After release of the N-terminally truncated forms of Aβ, such as Aβ3-x, i.e. Aβ3-40, and Aβ3-42, from APP or a fragment thereof, the enzyme glutaminyl cyclase (QC) is capable to catalyze the formation of pyroglutamate at the N-terminus of these truncated Aβ peptides resulting in the release of Aβ N3pE-x, e.g. Aβ N3pE-40 and Aβ N3pE-42. Consequently, the present invention describes for the first time a pathway, how highly toxic forms of Aβ, i.e. the N-terminally truncated and pyroGlu modified Aβ N3pE-x peptides, e.g. Aβ N3pE-40 and Aβ N3pE-42, may be formed from APP or APP fragments by the action of endopeptidases, which comprise meprin-α and/or meprin-β, together with the subsequent action of glutaminyl cyclase.

The prevention of the formation of the N-terminally truncated and pyroGlu modified Aβ N3pE-x peptides, e.g. Aβ N3pE-40 and Aβ N3pE-42 is most important for a successful prevention and/or treatment of Amyloidosis, in particular Alzheimer's disease and neurodegeneration in Down Syndrome.

Thus, the present invention relates also to antagonists of meprin-α and/or meprin-β, compositions comprising said antagonists and the use of said compositions for the treatment of amyloidosis, especially for the treatment of neurodegenerative disease in a mammal, in particular in a human. Said neurodegenerative disease is in particular selected from the group consisting of mild cognitive impairment (MCI), Alzheimer's disease (AD), like for instance sporadic Alzheimer's disease (SAD) or Familial Alzheimer's dementias (FAD) like Familial British Dementia (FBD) and Familial Danish Dementia (FDD), neurodegeneration in Down Syndrome. Preferably, said neurodegenerative disease is Alzheimer's disease.

In another embodiment of the invention, said composition comprises the antagonist in a therapeutically effective amount.

Further comprised by the invention is a mixture comprising at least one antagonist of meprin-α and/or meprin-β, and, optionally, a further biologically active substance and/or a pharmaceutically acceptable carrier and/or a diluent and/or an excipient.

In particular, the invention relates to a mixture, wherein the further biologically active substance is a compound used in the medication of amyloidosis, a group of diseases and disorders associated with amyloid or amyloid-like protein such as the Aβ protein involved in neurodegenerative diseases selected from the group consisting of mild cognitive impairment (MCI), Alzheimer's disease (AD), like for instance sporadic Alzheimer's disease (SAD) or Familial Alzheimer's dementias (FAD) like Familial British Dementia (FBD) and Familial Danish Dementia (FDD), neurodegeneration in Down Syndrome; preferably Alzheimer's disease.

Suitably, the other biologically active substance or compound may also be a therapeutic agent that may be used in the treatment of amyloidosis caused by amyloid β or may be used in the medication of other neurological disorders.

The other biologically active substance or compound may exert its biological effect by the same or a similar mechanism as antagonist of meprin-α and/or meprin-β according to the invention or by an unrelated mechanism of action or by a multiplicity of related and/or unrelated mechanisms of action.

Generally, the other biologically active compound may include neutron-transmission enhancers, psychotherapeutic drugs, acetylcholine esterase inhibitors, calcium-channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-D-aspartate glutamate receptor antagonists, non-steroidal anti-inflammatory drugs, antioxidants, and serotonergic receptor antagonists.

More particularly, the invention relates to a mixture comprising at least one compound selected from the group consisting of compounds effective against oxidative stress, anti-apoptotic compounds, metal chelators, inhibitors of DNA repair such as pirenzepin and metabolites, 3-amino-1-propanesulfonic acid (3 APS), 1,3-propanedisulfonate (1,3 PDS), α-secretase activators, β- and γ-secretase inhibitors, tau proteins, neurotransmitter, β-sheet breakers, attractants for amyloid beta clearing/depleting cellular components, inhibitors of N-terminal truncated amyloid beta including pyroglutamated amyloid beta 3-42, such as inhibitors of glutaminyl cyclase, anti-inflammatory molecules, or cholinesterase inhibitors (ChEIs) such as tacrine, rivastigmine, donepezil, and/or galantamine, Ml agonists and other drugs including any amyloid or tau modifying drug and nutritive supplements, and nutritive supplements, together with an antibody according to the present invention and, optionally, a pharmaceutically acceptable carrier and/or a diluent and/or an excipient.

The invention further relates to a mixture, wherein the compound is a cholinesterase inhibitor (ChEIs), particularly a mixture, wherein the compound is one selected from the group consisting of tacrine, rivastigmine, donepezil, galantamine, niacin and memantine.

In a further embodiment, the mixtures according to the invention may comprise niacin or memantine together with an antibody according to the present invention and, optionally, a pharmaceutically acceptable carrier and/or a diluent and/or an excipient.

In a further embodiment, the mixtures according to the invention may comprise a glutaminyl cyclase inhibitor together with an antibody according to the present invention and, optionally, a pharmaceutically acceptable carrier and/or a diluent and/or an excipient.

Preferred inhibitors of glutaminyl cyclase are described in WO 2005/075436, in particular examples 1-141 as shown on pp. 31-40. The synthesis of examples 1-141 is shown on pp. 40-48 of WO 2005/075436. The disclosure of WO 2005/075436 regarding examples 1-141, their synthesis and their use as glutaminyl cyclase inhibitors is incorporated herein by reference.

Further preferred inhibitors of glutaminyl cyclase are described in WO 2008/055945, in particular examples 1-473 as shown on pp. 46-155. The synthesis of examples 1-473 is shown on pp. 156-192 of WO 2008/055945. The disclosure of WO 2008/055945 regarding examples 1-473, their synthesis and their use as glutaminyl cyclase inhibitors is incorporated herein by reference.

Further preferred inhibitors of glutaminyl cyclase are described in WO 2008/055947, in particular examples 1-345 as shown on pp. 53-118. The synthesis of examples 1-345 is shown on pp. 119-133 of WO 2008/055947. The disclosure of WO 2008/055947 regarding examples 1-345, their synthesis and their use as glutaminyl cyclase inhibitors is incorporated herein by reference.

Further preferred inhibitors of glutaminyl cyclase are described in WO 2008/055950, in particular examples 1-212 as shown on pp. 57-120. The synthesis of examples 1-212 is shown on pp. 121-128 of WO 2008/055950. The disclosure of WO 2008/055950 regarding examples 1-212, their synthesis and their use as glutaminyl cyclase inhibitors is incorporated herein by reference.

Further preferred inhibitors of glutaminyl cyclase are described in WO2008/065141, in particular examples 1-25 as shown on pp. 56-59. The synthesis of examples 1-25 is shown on pp. 60-67 of WO2008/065141. The disclosure of WO2008/065141 regarding examples 1-25, their synthesis and their use as glutaminyl cyclase inhibitors is incorporated herein by reference.

Further preferred inhibitors of glutaminyl cyclase are described in WO 2008/110523, in particular examples 1-27 as shown on pp. 55-59. The synthesis of examples 1-27 is shown on pp. 59-71 of WO 2008/110523. The disclosure of WO 2008/110523 regarding examples 1-27, their synthesis and their use as glutaminyl cyclase inhibitors is incorporated herein by reference.

Further preferred inhibitors of glutaminyl cyclase are described in WO 2008/128981, in particular examples 1-18 as shown on pp. 62-65. The synthesis of examples 1-18 is shown on pp. 65-74 of WO 2008/128981. The disclosure of WO 2008/128981 regarding examples 1-18, their synthesis and their use as glutaminyl cyclase inhibitors is incorporated herein by reference.

Further preferred inhibitors of glutaminyl cyclase are described in WO 2008/128982, in particular examples 1-44 as shown on pp. 61-67. The synthesis of examples 1-44 is shown on pp. 68-83 of WO 2008/128982. The disclosure of WO 2008/128982 regarding examples 1-44, their synthesis and their use as glutaminyl cyclase inhibitors is incorporated herein by reference.

Further preferred inhibitors of glutaminyl cyclase are described in WO 2008/128983, in particular examples 1-30 as shown on pp. 64-68. The synthesis of examples 1-30 is shown on pp. 68-80 of WO 2008/128983. The disclosure of WO 2008/128983 regarding examples 1-30, their synthesis and their use as glutaminyl cyclase inhibitors is incorporated herein by reference.

Further preferred inhibitors of glutaminyl cyclase are described in WO 2008/128984, in particular examples 1-36 as shown on pp. 63-69. The synthesis of examples 1-36 is shown on pp. 69-81 of WO 2008/128984. The disclosure of WO 2008/128984 regarding examples 1-36, their synthesis and their use as glutaminyl cyclase inhibitors is incorporated herein by reference.

Further preferred inhibitors of glutaminyl cyclase are described in WO 2008/128985, in particular examples 1-71 as shown on pp. 66-76. The synthesis of examples 1-71 is shown on pp. 76-98 of WO 2008/128985. The disclosure of WO 2008/128985 regarding examples 1-71, their synthesis and their use as glutaminyl cyclase inhibitors is incorporated herein by reference.

Further preferred inhibitors of glutaminyl cyclase are described in WO 2008/128986, in particular examples 1-7 as shown on pp. 65-66. The synthesis of examples 1-7 is shown on pp. 66-73 of WO 2008/128986. The disclosure of WO 2008/128986 regarding examples 1-7, their synthesis and their use as glutaminyl cyclase inhibitors is incorporated herein by reference.

Further preferred inhibitors of glutaminyl cyclase are described in WO 2008/128987, in particular examples 1-12 as shown on pp. 58-59. The synthesis of examples 1-12 is shown on pp. 60-63 of WO 2008/128987. The disclosure of WO 2008/128987 regarding examples 1-12, their synthesis and their use as glutaminyl cyclase inhibitors is incorporated herein by reference.

Further preferred inhibitors of glutaminyl cyclase are described in WO 2010/026212, in particular examples 1-46 as shown on pp. 77-84. The synthesis of examples 1-46 is shown on pp. 84-99 of WO 2010/026212. The disclosure of WO 2010/026212 regarding examples 1-46, their synthesis and their use as glutaminyl cyclase inhibitors is incorporated herein by reference.

In still another embodiment of the invention mixtures are provided that comprise “atypical antipsychotics” such as, for example clozapine, ziprasidone, risperidone, aripiprazole or olanzapine for the treatment of positive and negative psychotic symptoms including hallucinations, delusions, thought disorders (manifested by marked incoherence, derailment, tangentiality), and bizarre or disorganized behavior, as well as anhedonia, flattened affect, apathy, and social withdrawal, together with an antagonist of meprin-α and/or meprin-β according to the invention and as described herein and, optionally, a pharmaceutically acceptable carrier and/or a diluent and/or an excipient.

Other compounds that can be suitably used in mixtures in combination with the antagonist of meprin-α and/or meprin-β according to the present invention are described in WO2008/065141 (see especially pages 37/38), including PEP-inhibitors (pp. 43/44), LiCl, inhibitors of dipeptidyl aminopeptidases, preferably inhibitors of DP IV or DP IV-like enzymes (see pp. 48/49); acetylcholinesterase (ACE) inhibitors (see p. 47), PIMT enhancers, inhibitors of beta secretases (see p. 41), inhibitors of gamma secretases (see pp. 41/42), inhibitors of neutral endopeptidase, inhibitors of phosphodiesterase-4 (PDE-4) (see pp. 42/43), TNFalpha inhibitors, muscarinic M1 receptor antagonists (see p. 46), NMDA receptor antagonists (see pp. 47/48), sigma-1 receptor inhibitors, histamine H3 antagonists (se p. 43), immunomodulatory agents, immunosuppressive agents or an agent selected from the group consisting of antegren (natalizumab), Neurelan (fampridine-SR), campath (alemtuzumab), IR 208, NBI 5788/MSP 771 (tiplimotide), paclitaxel, Anergix.MS (AG 284), SH636, Differin (CD 271, adapalene), BAY 361677 (interleukin-4), matrix-metalloproteinase-inhibitors (e.g. BB 76163), interferon-tau (trophoblastin) and SAIK-MS; beta-amyloid antibodies (see p. 44), cysteine protease inhibitors (see p. 44); MCP-1 antagonists (see pp. 44/45), amyloid protein deposition inhibitors (see 42) and beta amyloid synthesis inhibitors (see p. 42), which document is incorporated herein by reference.

In another embodiment, the invention relates to a mixture comprising the antagonist of meprin-α and/or meprin-β according to the present invention and as described herein before and/or the further biologically active substance in a therapeutically effective amount.

Suitably, the invention relates to a mixture comprising the antagonist of meprin-α and/or meprin-β according to the present invention and as described herein before, wherein said antagonist is an inhibitor of meprin-α and/or meprin-β. Most suitably, said antagonist is an inhibitor of meprin-β.

The invention further relates to the use of an antagonist of meprin-α and/or meprin-β, or a pharmaceutical composition or a mixture comprising said antagonist of meprin-α and/or meprin-β, for the preparation of a medicament for treating or alleviating the effects of amyloidosis, a group of diseases and disorders associated with amyloid plaque formation including secondary amyloidosis and age-related amyloidosis such as diseases including, but not limited to, neurological disorders such as Alzheimer's Disease (AD), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, and others, including macular degeneration.

The invention further relates to methods of treatment preventing, treating or alleviating the effects of amyloidosis. In particular, the invention relates to methods of preventing, treating or alleviating of a disease selected from the group consisting of diseases and disorders associated with amyloid plaque formation including secondary amyloidosis and age-related amyloidosis such as diseases including, but not limited to, neurological disorders such as Alzheimer's Disease (AD), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, and others, including macular degeneration, comprising administering to a subject in need thereof at least one antagonist of meprin-α and/or meprin-β, or a pharmaceutical composition or a mixture comprising said antagonist of meprin-α and/or meprin-β.

Suitably, the invention relates to the use of an antagonist of meprin-α and/or meprin-β, or a method of treatment, or a pharmaceutical composition or a mixture comprising said antagonist of meprin-α and/or meprin-β, wherein said antagonist is an inhibitor of meprin-α and/or meprin-β. Most suitably, said antagonist is an inhibitor of meprin-β.

### Pharmaceutical Compositions

To prepare the pharmaceutical compositions of this invention, at least one antagonist of meprin-α and/or meprin-β in combination with at least one of the other aforementioned agents can be used as the active ingredient(s). The active ingredient(s) is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included.

Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient(s) necessary to deliver an effective dose as described above. The pharmaceutical compositions herein will contain, per dosage unit, e.g., tablet, capsule, powder, injection, suppository, teaspoonful and the like, from about 0.03 mg to 100 mg/kg (preferred 0.1-30 mg/kg) and may be given at a dosage of from about 0.1-300 mg/kg per day (preferred 1-50 mg/kg per day) of each active ingredient or combination thereof. The dosages, however, may be varied depending upon the requirement of the patients, the severity of the condition being treated and the compound being employed. The use of either daily administration or post-periodic dosing may be employed.

Suitably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of each active ingredient or combinations thereof of the present invention.

The tablets or pills of the compositions of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

This liquid forms in which the compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinylpyrrolidone or gelatin.

The pharmaceutical composition may contain between about 0.01 mg and 100 mg, suitably about 5 to 50 mg, of each compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or betalactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitable flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compounds or combinations of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

Compounds or combinations of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamid-ephenol, or polyethyl eneoxidepolyllysine substituted with palmitoyl residue. Furthermore, the compounds of the present invention may be coupled to a class of biodegradable polymers useful in achieving controlled release of a drug, for example, polylactic acid, polyepsilon caprolactone, polyhydroxy butyeric acid, polyorthoesters, polyacetals, polydihydropyrans, polycyanoacrylates and cross-linked or amphipathic block copolymers of hydrogels.

Compounds or combinations of this invention may be administered in any of the foregoing compositions and according to dosage regimens established in the art whenever treatment of the addressed disorders is required.

The daily dosage of the products may be varied over a wide range from 0.01 to 1.000 mg per mammal per day. For oral administration, the compositions are suitably provided in the form of tablets containing, 0.01, 0.05, 0.1, 0.5, 1.0, 2.5, 5.0, 10.0, 15.0, 25.0, 50.0, 100, 150, 200, 250 and 500 milligrams of each active ingredient or combinations thereof for the symptomatic adjustment of the dosage to the patient to be treated. An effective amount of the drug is ordinarily supplied at a dosage level of from about 0.1 mg/kg to about 300 mg/kg of body weight per day. Suitably, the range is from about 1 to about 50 mg/kg of body weight per day. The compounds or combinations may be administered on a regimen of 1 to 4 times per day.

Optimal dosages to be administered may be readily determined by those skilled in the art, and will vary with the particular compound used, the mode of administration, the strength of the preparation, the mode of administration, and the advancement of disease condition. In addition, factors associated with the particular patient being treated, including patient age, weight, diet and time of administration, will result in the need to adjust dosages.

In a further aspect, the invention also provides a process for preparing a pharmaceutical composition comprising at least one compound of formula (I), optionally in combination with at least one of the other aforementioned agents and a pharmaceutically acceptable carrier.

The compositions are suitably in a unit dosage form in an amount appropriate for the relevant daily dosage.

Suitable dosages, including especially unit dosages, of the compounds of the present invention include the known dosages including unit doses for these compounds as described or referred to in reference text such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein) or the above mentioned publications.

### Screening Assays

In another aspect, the present invention provides screening assays for identifying antagonists (inhibitors) of the ability of meprin-α and/or meprin-β to interfere with APP amyloidogenic processing resulting in the modulation of Aβ production. Meprin-α and/or meprin-β, APP and processing secretases, any or all of which may be present in isolated, immobilized or cell bound form or in the form of membrane-enzyme mixture, are contacted with a candidate compound, or a plurality of candidate compounds, and those candidates are selected that alter, preferably inhibit, meprin-α and/or meprin-β mediated Aβ production. While the effect of a candidate compound on meprin-α and/or meprin-β activity is preferably detected by monitoring its ability to alter (e.g. decrease) the amount of Aβ produced, other read-outs of APP or APP fragments cleavage at the α, β or γ-secretase cleavage sites are equally suitable. Both cell-free and cell based assays, including assays performed with cell membrane-enzyme preparations are specifically within the scope of the invention.

Suitably, screening methods are provided, wherein the production of full-length Aβ polypeptides, such as Aβ1-40 or Aβ1-42, is monitored.

More suitably, the production of N-terminally truncated forms of Aβ, such as Aβ3-x, i.e. Aβ3-40, and Aβ3-42, is monitored.

Even more suitably, screening methods are provided, wherein only meprin-β is employed.

Candidate compounds which significantly reduce the ability of meprin-α and/or meprin-β to promote APP cleavage at or around the β-secretase cleavage site are preferred. Such compounds preferably inhibit the ability of meprin-α and/or meprin-β to produce full-length Aβ polypeptides, such as Aβ1-40 or Aβ1-42 from APP. More preferably, candidate compounds inhibit the ability of meprin-α and/or meprin-β to produce N-terminally truncated forms of Aβ, such as Aβ3-x, i.e. Aβ3-40, and Aβ3-42.

Particularly preferred candidate compounds reduce the level of said Aβ peptides by at least about 25%, preferably at least about 50%, more preferably at least about 75%, most preferably at least about 90%, and often at least about 95%. The compounds identified can be used in the treatment of patients, particularly humans, at risk of developing or diagnosed with amyloidosis, in particular AD, AD-type pathologies, cerebral amyloid angiopathy or any other pathology associated with the formation of β-amyloid deposits (e.g. plaques) in the CNS, such as brain.

The compounds of the invention encompass numerous chemical classes, including but not limited to the compounds described herein with known function. Novel methods are provided which employ compounds that are effective in inhibiting meprin-α and/or meprin-β mediated Aβ production.

Candidate compounds can be obtained from a wide variety of sources including libraries of synthetic or natural compounds. For example, numerous means are available for random and directed synthesis of a wide variety of organic compounds and biomolecules, including expression of randomized oligonucleotides and oligopeptides.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological compounds may be subjected to directed or random chemical modifications, such as acylation, alkylation, esterification, amidification, etc. to produce structural analogs.

Compounds for use in the method of invention may be small organic compounds having a molecular weight of more than 50 and less than about 2,500 daltons. Candidate compounds comprise functional groups necessary for structural interaction with proteins, particularly hydrogen bonding, and typically include at least an amine, carbonyl, hydroxyl or carboxyl group, preferably at least two of the functional chemical groups. The candidate compounds often comprise cyclical carbon or heterocyclic structures and/or aromatic or polyaromatic structures substituted with one or more of the above functional groups. Candidate compounds are also found among biomolecules including, but not limited to: peptides, saccharides, fatty acids, steroids, purines, pyrimidines, derivatives, structural analogs or combinations thereof.

Suitably, candidate compounds of the present invention are antagonists of meprin α and/or meprin β. More suitably, candidate compounds of the present invention are inhibitors of meprin α and/or meprin β. Most suitably, candidate compounds of the present invention are inhibitors of meprin β.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Degradation of APP-Derived Substrates Containing the β-Secretase Cleavage Site by Meprin α and β

### Substrates

According to the sequence of human APP, peptide substrates containing the β-secretase site were synthesized. The substrates differed and contained various mutations (the numbering refers to hAPP₆₉₅: swedish mutation KM595/596NL and artificial mutations E599Q and D597isoD) at or close to the β-secretase cleavage site. The substrate sequences are listed below:

wildtyp (wt)
(SEQ ID NO: 7)
H-GLTNIKTEEISEVKMDAEFRHDSGYEVHHQ-NH₂
E599Q
(SEQ ID NO: 8)
H-GLTNIKTEEISEVKMDAQFRHDSGYEVHHQ-NH₂
D597isoD
(SEQ ID NO: 9)
H-GLTNIKTEEISEVKMiDAEFRHDSGYEVHHQ-NH₂
swedish (sw)
(SEQ ID NO: 10)
H-GLTNIKTEEISEVNLDAEFRHDSGYEVHHQ-NH₂
sw E599Q
(SEQ ID NO: 11)
H-GLTNIKTEEISEVNLDAQFRHDSGYEVHHQ-NH₂
sw D597isoD
(SEQ ID NO: 12)
H-GLTNIKTEEISEVNLiDAEFRHDSGYEVHHQ-NH₂

Recombinant human meprin α and β were purchased from R&D systems. The peptide cleavage of the substrates by both meprins was investigated in 85 mM Tris/HCl buffer, pH 7.5.

Typically, 25 μl of substrate in buffer (200 μM) was added to 15 μl of water, 50 μl buffer and 10 μl enzyme (30-60 nM). A 5 μl sample was taken every 2 hours, mixed with 5 μl sinapinic acid and analyzed by MALDI-TOF mass spectrometry.

### Results

### Meprin-Mediated Cleavage of APP-Derived Peptides

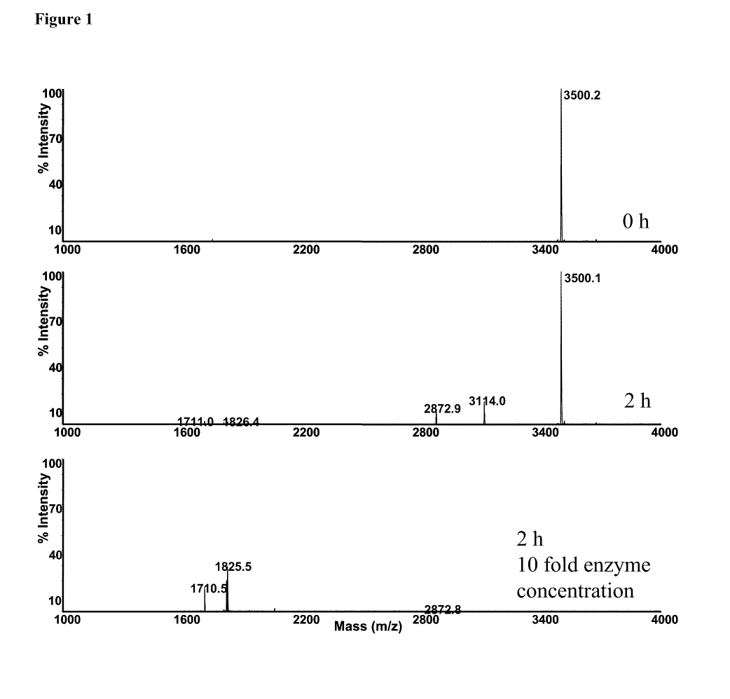

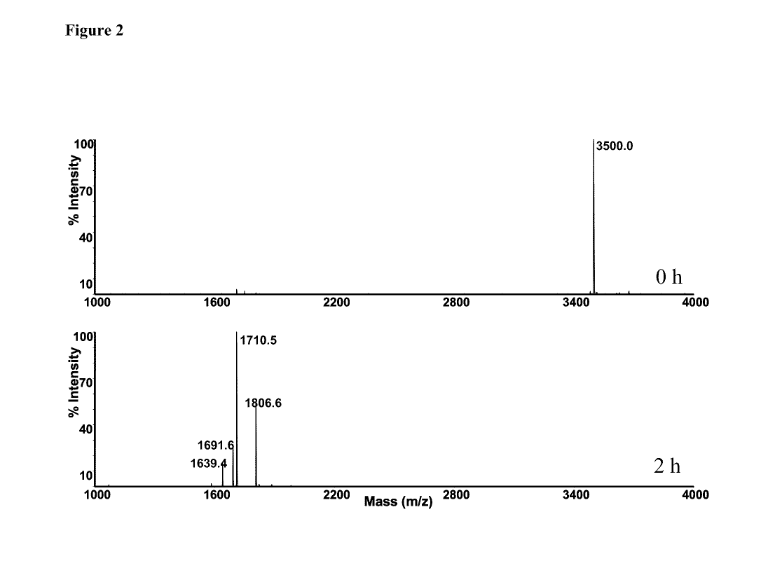

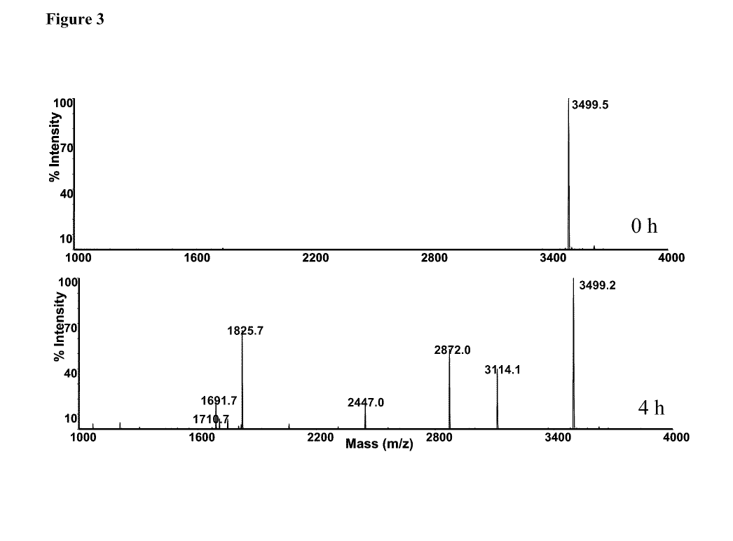

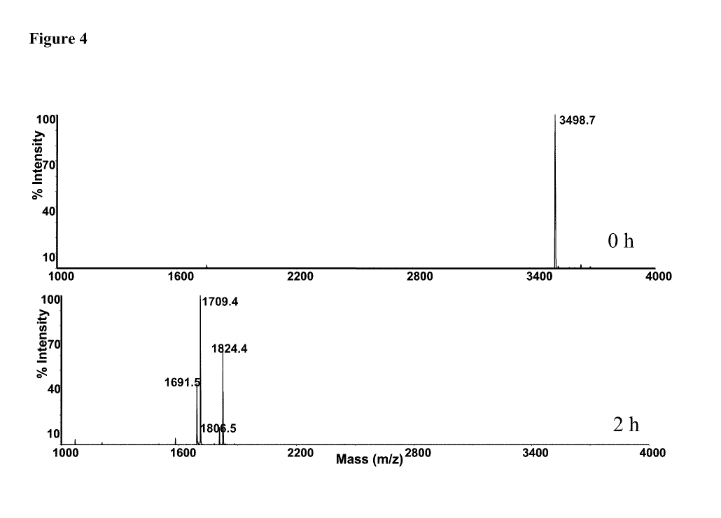

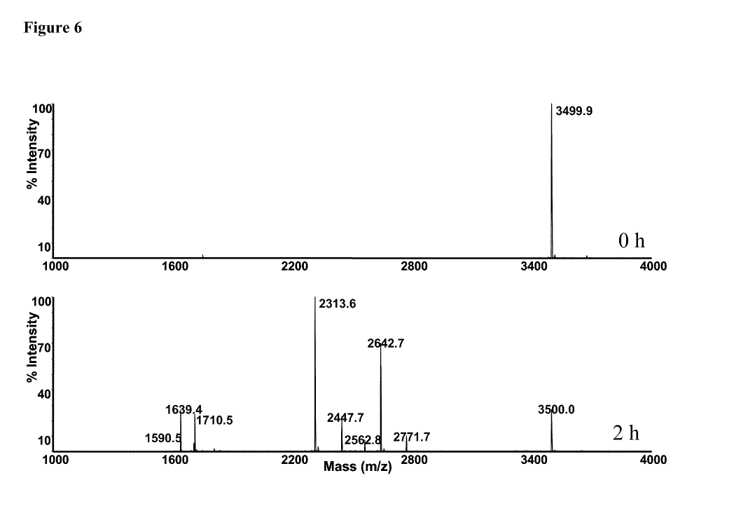

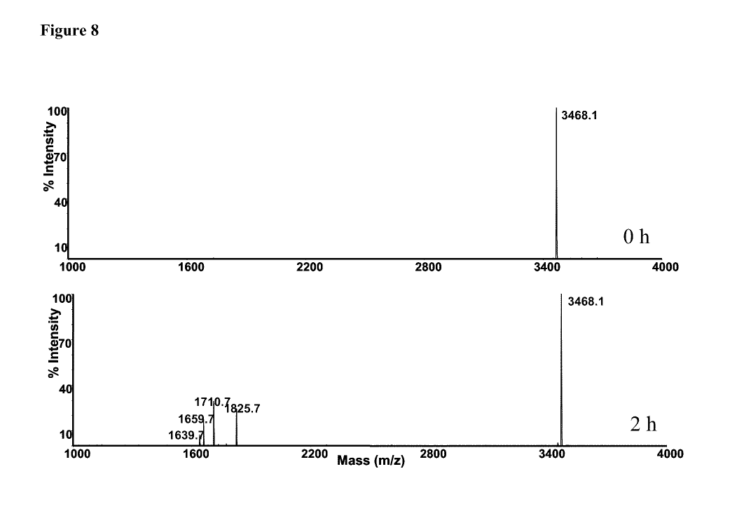

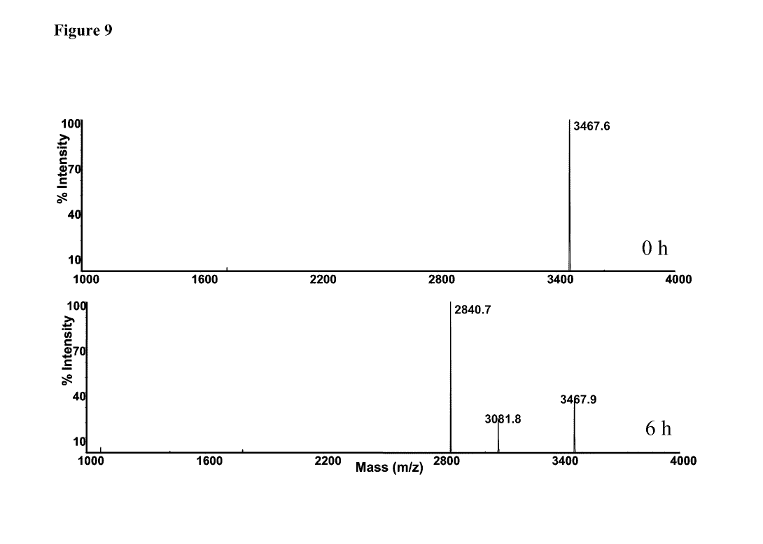

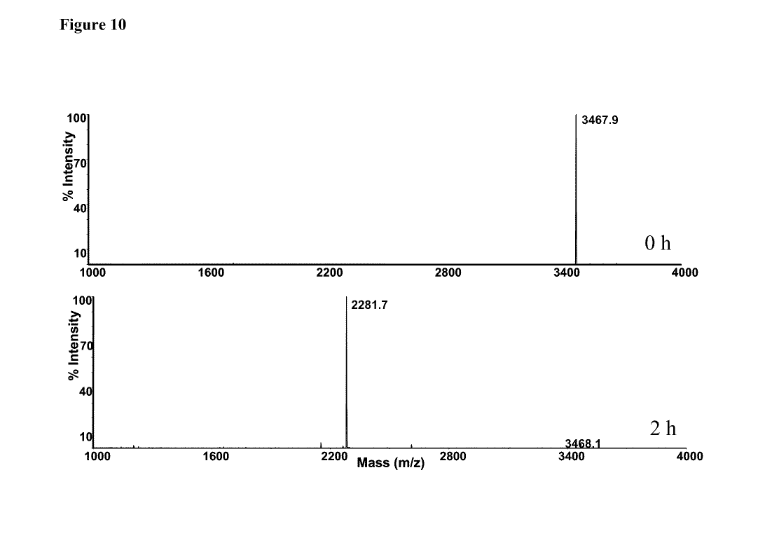

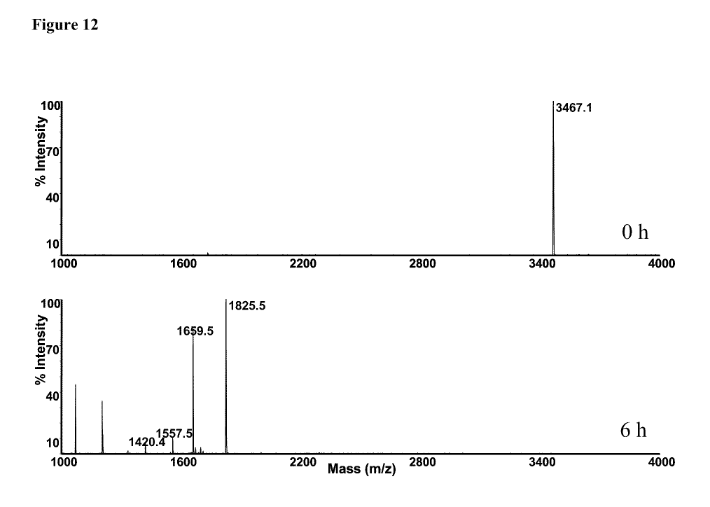

In the following, the peptide fragments (numbering according to substrate 1-30) of Aβ are listed according to the substrate used. The list relates to the FIGS. 1-12. An overview of the results is provided in Table 2.

wt and D597isoD [M+H⁺]3500.85 (see FIGS. 1,2 for wt and 3,4 for D597isoD)

Fragments generated by the meprin α or meprin β subunits:

| | |
|---|---|
| [M + H⁺] 3114.73 | 5-30 |
| [M + H⁺] 2873.09 | 7-30 |
| [M + H⁺] 2447.0 | 1-21 |
| [M + H⁺] 1710.80 | 17-30 |
| [M + H⁺] 1825.89 | 16-30 |
| [M + H⁺] 1806.08 | 1-16 |
| [M + H⁺] 1690.99 | 1-15 |
| [M + H⁺] 1639.72 | 18-30 |

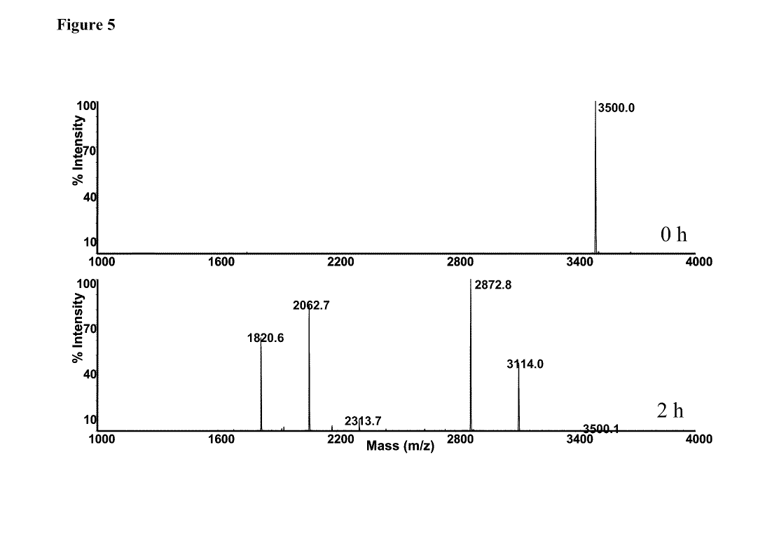

### E599Q [M+H*]3499.86 (see FIGS. 5,6)

Fragments generated by meprin α or meprin β subunits:

| | |
|---|---|
| [M + H⁺] 3113.44 | 5-30 |
| [M + H⁺] 2872.11 | 7-30 |
| [M + H⁺] 2771.00 | 8-30 |
| [M + H⁺] 2641.88 | 9-30 |
| [M + H⁺] 2561.88 | 1-22 |
| [M + H⁺] 2446.79 | 1-21 |
| [M + H⁺] 2312.53 | 12-30 |
| [M + H⁺] 2060.38 | 5-21 |
| [M + H⁺] 1819.04 | 7-21 |
| [M + H⁺] 1709.81 | 17-30 |
| [M + H⁺] 1638.74 | 18-30 |
| [M + H⁺] 1589.80 | 9-21 |

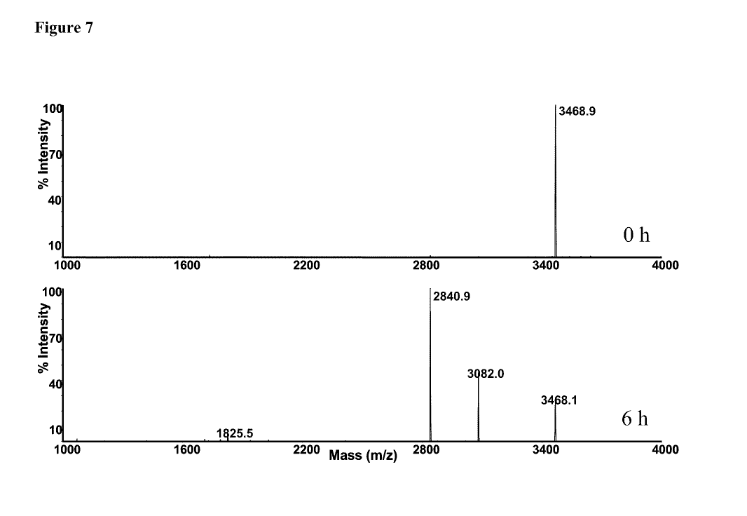

sw and swD597isoD [M+H⁺]3468.74 (see FIGS. 7,8 for sw and 9,10 for swD597isoD)

Fragments generated by meprin α or meprin β subunits:

| | |
|---|---|
| [M + H⁺] 3082.32 | 5-30 |
| [M + H⁺] 2840.98 | 7-30 |
| [M + H⁺] 2281.40 | 12-30 |
| [M + H⁺] 1825.89 | 16-30 |
| [M + H⁺] 1710.80 | 17-30 |
| [M + H⁺] 1659.87 | 1-15 |
| [M + H⁺] 1639.72 | 18-30 |
| [M + H⁺] 1512.57 | 18-29 |

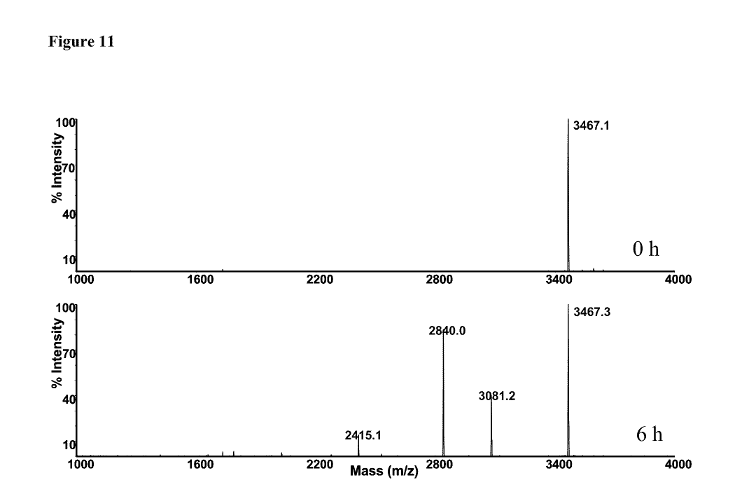

### sw E599Q [M+H*]3467.76 (see FIGS 11,12)

Fragments generated by meprin α or meprin β subunits:

| | |
|---|---|
| [M + H⁺] 3081.33 | 5-30 |
| [M + H⁺] 2840.00 | 7-30 |
| [M + H⁺] 2414.68 | 1-21 |
| [M + H⁺] 1825.90 | 16-30 |
| [M + H⁺] 1659.87 | 1-15 |
| [M + H⁺] 1557.70 | 9-21 |
| [M + H⁺] 1420.55 | 9-20 |

Summarizing the results (Table 2), it becomes obvious that cleavage of the peptides by meprin α or meprin β subunits leads to generation of peptides with an N-terminus as known for full-length or N-truncated Aβ peptides. Most importantly, a cleavage at position 18 occurs (corresponding to Aβ 3-x) by meprin β, which generates the precursor of pGlu-modified Aβ.

Concluding, both, the meprin α and meprin β subunits display a cleavage pattern which is expected from a β-secretase and thus contribute to the generation of Aβ in vivo.

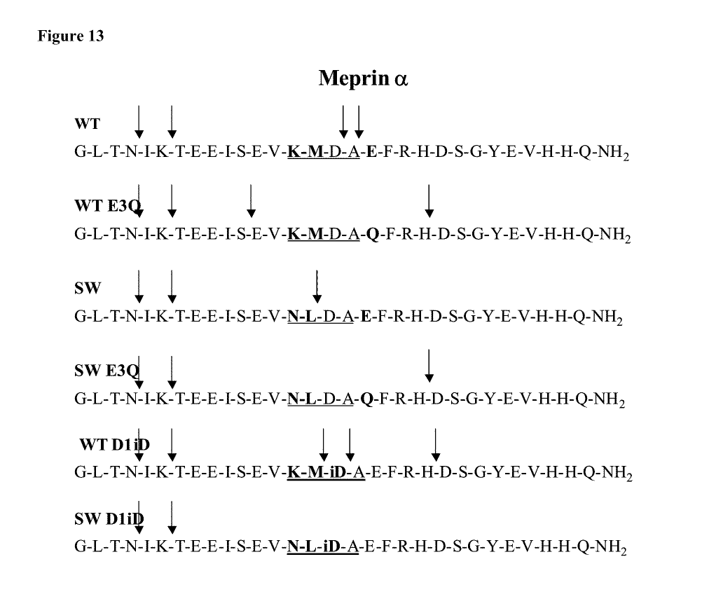

The most important finding relates to the fact that meprin cleavage directly leads to generation of a truncated N-terminus of Aβ The cleavage pattern is further illustrated in FIG. 13.

TABLE 2
Found degradation products after incubation of substrates with meprin
α or meprin β subunits (numbering according to substrate H-
GLTNIKTEEISEVxyzAnFRHDSGYEVHHQ-NH₂).

| | | wt | wt | | sw | sw |
|---|---|---|---|---|---|---|
| | wt | E599Q | D597isoD | sw | E599Q | D597isoD |
| MEP A | 5-30 | 5-30 | 5-30 | 5-30 | 5-30 | 5-30 |
| | 7-30 | 7-30 | 7-30 | 7-30 | 7-30 | 7-30 |
| | 17-30 | 5-21 | 1-21 | 16-30 | 1-21 | |
| | 16-30 | 7-21 | 16-30 | | | |
| | | 12-30 | 17-30 | | | |
| | | | 1-15 | | | |
| MEP B | 17-30 | 12-30 | 1-15 | 18-30 | 16-30 | 12-30 |
| | 1-16 | 9-30 | 17-30 | 1-15 | 1-15 | |
| | 1-15 | 9-21 | 16-30 | 16-30 | 9-21 | |
| | 18-30 | 1-22 | 1-16 | 17-30 | 9-20 | |
| | | 1-21 | | | | |
| | | 8-30 | | | | |
| | | 18-30 | | | | |
| | | 17-30 | | | | |

### Example 2

### Generation of a Stable Cell Line Expressing Human APP (HEK293)

A prerequisite for testing of the β-secretase-activity of meprins in cell culture is the generation of cell lines, which preferably express the substrate human APP stably.

Accordingly, the sequence of human APP was cloned into the vector pIRESneo (Clontech). The resulting clone was named pIRES-hAPP. For overexpression of hAPP the cells were cultured in 6-well dishes until 60% confluence, transfected with the generated vector or the empty vector (1 μg DNA/well) using Lipofectamin2000 (Invitrogen) according to manufacturer's manual and incubated in the transfection solution for 20 hours. Afterwards, the solution was replaced by appropriate growth media supplemented with G418 (80 ng/ml) for selection. The medium was replaced every 24 hours. After 2 weeks, remaining colonies of G418 insensitive cells were split and diluted for single cell cloning. Single cells were grown in 96-well plates. Colonies were proliferated and investigated for APP expression by western blotting using an antibody directed against human APP. Selected clones were further investigated by qPCR for Expression of hAPP mRNA. For investigation of secreted Aβ, clones were spread into 6-well plates. After 24 h, the culture medium was replaced by serum- and phenolred-free medium. The medium was removed again 24 h later, supplemented with a protease inhibitor cocktail (complete, Mini, Roche), centrifuged at 500×g and 10 000×g subsequently to remove cells and debris. The supernatant was analyzed using an Aβx-40 ELISA (IBL) according to manufacturer's manual for Aβ secretion.

### Results

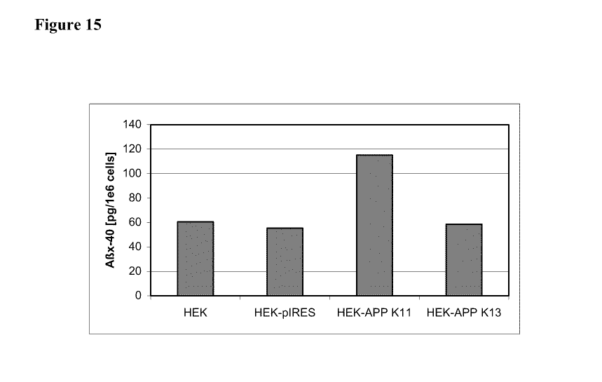

Clone 11 and 13 have been shown by qPCR to produce elevated levels of mRNA of hAPP (Table 3). Clone 11 displayed a doubled secretion of Aβx-40, which is due to cleavage by 3-secretases expressed by HEK293 cells, e.g. β-secretase BACE (FIG. 15). The cell line will be employed to test the expression of meprin α and meprin β on the secretion of Aβ.

For that purpose, cells will be transiently transfected with a plasmid encoding meprin-A and meprin-B and the medium will be analyzed as described before using ELISA techniques (e.g. Aβ ELISA from IBL international, Hamburg, Germany) to proof an increase in Aβ-production upon overexpression of meprin α and meprin β.

TABLE 3
Increase (fold increase) of APP mRNA in HEK293
cells after stable transfection with pIRES-hAPP.

| cell line | fold increase of APP mRNA |
|---|---|
| HEK293 | 1.00 |
| HEK-pIRES | 1.33 |
| HEK-hAPP K11 | 3.46 |
| HEK-hAPP K13 | 4.76 |

### Example 3

### Transient Overexpression of Human Meprin α and β in HEK—Influence on Secreted Aβ

The sequence of human meprin α and β were cloned into the vector pcDNA3.1 (Invitrogen). The resulting clone was named pcDNA-hMPβ and pcDNA-hMPα, respectively.

For transient overexpression of human meprin the cells were cultured in 6-well dishes until 90% confluence, transfected with 2 μg of the generated or empty vector per well using Lipofectamin2000 (Invitrogen) according to manufacturer's manual. After 24 h the solution was replaced by medium free of serum and phenolred. Medium was collected 24 h later and supplemented with a protease inhibitor cocktail (complete, Mini, Roche), centrifuged at 500×g and 10 000×g, subsequently to remove cells and debris. The supernatant was stored at −80 until analysis by ELISA for Aβx-40.

### Results

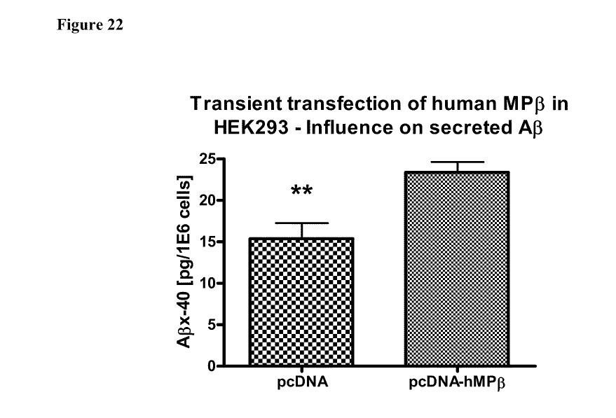

After transient transfection of HEK293 cells with pcDNA-hMPβ amount of secreted Aβ (detected with ELISA from IBL, specific for Aβ40, non specific for the Aβ N-terminus, FIG. 22) is significantly increased (see FIG. 22).

### Example 4

### Transient Overexpression of Human Meprin and Human APP (Wildtyp and Mutated Forms) in HEK293

The sequence of human APP was cloned into the vector pcDNA3.1 (Invitrogen) and various mutations were inserted. The resulting clones were named pcDNA-hAPP (for wildtyp APP), pcDNA-hAPPsw (for mutation K595N, M596L) and pcDNA-hAPP/E3Q (for mutation E599Q). For transient overexpression of human meprin and human APP the cells were cultured in 6-well dishes until 90% confluence, transfected with 2 μg of vector with meprin and 2 μg of one of the described APP-vectors per well using Lipofectamin2000 (Invitrogen) according to manufacturer's manual. Control samples were transfected with pcDNA3.1. After 24 h the solution was replaced by medium free of serum and phenolred. Medium was collected 24 h later and supplemented with a protease inhibitor cocktail (complete, Mini, Roche), centrifuged at 500×g and 10 000×g, subsequently to remove cells and debris. The supernatant was stored at −80 until analysis by ELISA (IBL) and urea western-blot for identification of liberated Aβ species.

### Results

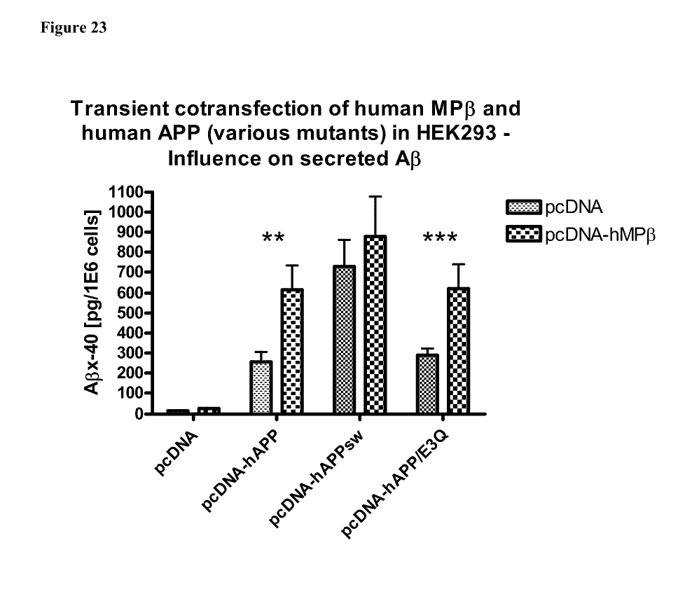

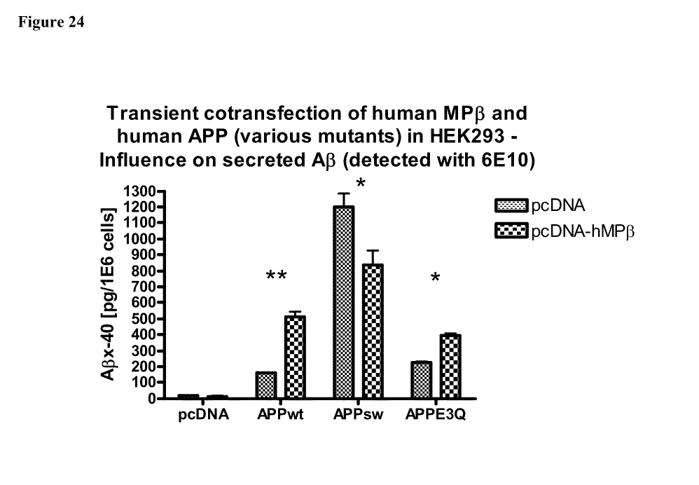

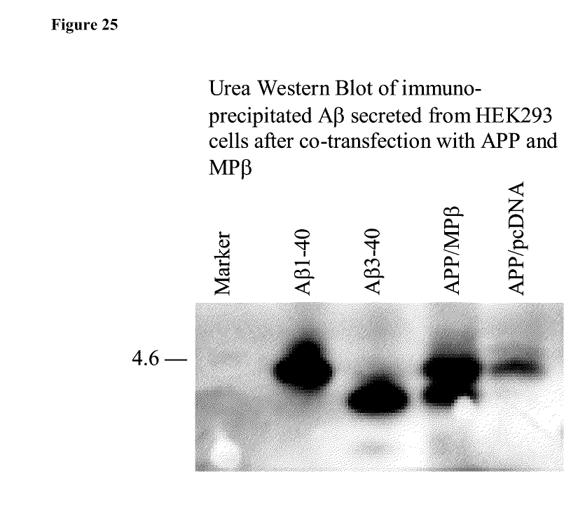

After transient transfection of HEK293 cells with pcDNA-hMPβ and APP-constructs amount of secreted Aβ (detected with ELISA from IBL, non specific for the Aβ N-terminus) is significantly increased for APPwt and APP/E3Q (FIG. 23). Quantification of secreted Aβ using antibody 6E10 instead of that supplied with the kit confirmed the results for APPwt and APP/E3Q (FIG. 24). The difference in secreted Aβ with and without MPβ became significant for APPsw, too. 6E10 specifically detects Aβ starting at position 1 to 7. So a possible function of MPβ as α-secretase could be excluded. A further characterization of generated Aβ species by urea western-blot (see FIG. 25) revealed the formation of an additional Aβ species after co-transfection with hAPP and human meprin β compared to transfection with hAPP. This additional Aβ species co-migrates with Aβ3-40.

### Example 5

### Inhibition of Activity of Meprin after Transient Overexpression of Human Meprin and Human APP in HEK293—Influence on Secreted Aβ

Activity of meprin can be inhibited by various inhibitors of matrix metalloproteases, for example Actinonin, Batimastat, Galardin, NN-GH and PLG-NHOH (Kruse, M.-N. et al., Biochem. J. (2004), 378, pp. 383-389)(incorporated herein by reference). Therefore application of these inhibitors after overexpression of APP and MP in KEK293 should have a reversible effect on the increase of Aβ secretion.

24 h after co-transfection of HEK293 cells with human APP and meprin (as described for example 4) the medium was replaced by medium free of serum and phenolred supplemented with 20 μM Actinonin (1% DMSO). 24 hours later medium was collected, supplemented with inhibitor cocktail (complete, Mini, Roche), centrifuged at 500×g and 10 000×g, subsequently to remove cells and debris. The supernatant was stored at −80 until analysis by ELISA (IBL).

### Results

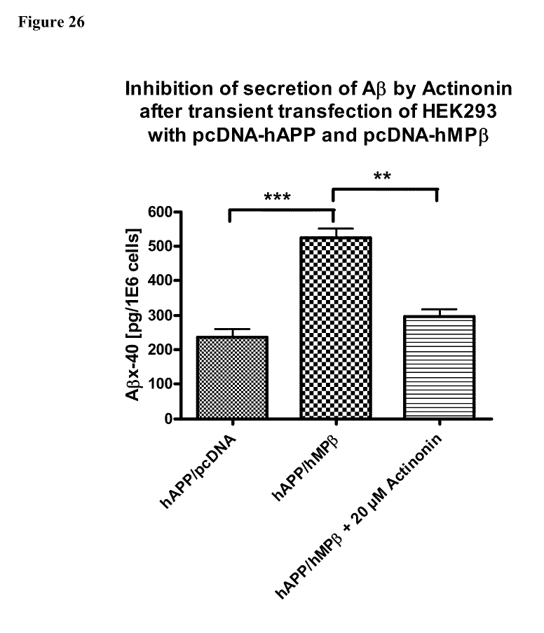

Secretion of Aβ is significantly increased in HEK293 after transient overexpression of hAPP and hMPβ (see FIG. 26). This increase is reduced to almost the basal level by the addition of 20 μM actinonin. To discriminate between effects of actinonin on α-secretases and β-secretases the antibody in the ELISA-kit (IBL) was replaced by antibody 6E10.

### Example 6

### Determination of Inhibitor Constants

Meprin activities were determined using N-benzoyl-L-tyrosyl-p-aminobenzoic acid as substrate. The substrate concentration was 40 mM, and the enzyme concentration was already at least 10 times below K. Inhibitors were employed in a concentration range from 5 pM to 5 mM. Each inhibitor was tested over a concentration range covering at least ten different concentrations from Kᵢ/5 to 5×Kᵢ. All reactions were carried out at 37° C. in 50 mM Tris/HCl, pH 7.5, and 0.5 mM MgCl₂.

Determination of the inhibition constant Kᵢ was performed by non-linear regression analysis using GraFit 4.0 by plotting the ratio of the inhibited and uninhibited enzyme activities against the inhibitor concentration. In the case of weak inhibition IC50 values were obtained from plots of the relative activity against −log [I] and calculated using GrafFit 4.0.

What is claimed is:

1. A method of modulating the enzymatic production of β-amyloid peptide (Aβ) from β-amyloid precursor protein (APP) or a fragment thereof, comprising:
contacting said APP or APP fragment with (i) a meprin-α or a meprin-β polypeptide; or
(ii) an antagonist of meprin-α or meprin-β.

2. The method of claim 1, wherein said APP is a native sequence human APP.

3. The method of claim 1, wherein said APP is the 695-amino acid isotype.

4. The method according to claim 1, wherein said APP comprises a Swedish mutation of K595N and M596L.

5. The method according to claim 1, wherein said meprin-α is a native sequence meprin-α polypeptide.

6. The method according to claim 1, wherein said meprin-β is a native sequence meprin-β polypeptide.

7. A method of inhibiting the formation of a β-amyloid peptide (Aβ) from β-amyloid precursor protein (APP) or a fragment thereof, comprising:
contacting said APP or APP fragment with an antagonist of meprin-α or meprin-β.

8. The method of claim 7, wherein said APP is a native sequence human APP.

9. The method of claim 7, wherein said APP is the 695-amino acid isotype.

10. The method of claim 7, wherein APP comprises a Swedish mutation of K595N and M596L.

11. The method of claim 7, wherein said antagonist inhibits the formation of a β-amyloid peptide (Aβ) from β-amyloid precursor protein (APP) or a fragment thereof, which is catalyzed by meprin-α.

12. The method of claim 7, wherein said antagonist inhibits the formation of a β-amyloid peptide (Aβ) from β-amyloid precursor protein (APP) or a fragment thereof, which is catalyzed by meprin-β.

13. The method of claim 7, wherein in the method is performed in the presence of an α-secretase activity.

14. The method of claim 7, wherein the method is performed in the presence of an γ-secretase activity.

15. The method of claim 7, wherein the method is performed in the presence of a β-secretase activity other than meprin-α or meprin-β.

16. The method of claim 15, wherein the method is performed in the presence of BACE1 or BACE2.

17. The method of claim 7, wherein said meprin-α or meprin-β is in isolated form.

18. The method of claim 7, wherein said meprin-α or meprin-β is in immobilized or cell bound form.

19. The method of claim 7, wherein said APP or APP fragment is contacted with an antagonist of meprin-α.

20. The method of claim 7, wherein said APP or APP fragment is contacted with an antagonist of meprin-β.

21. The method of claim 7, wherein said antagonist of meprin-α or meprin-β is an inhibitor.

22. The method of claim 7, wherein said antagonist of meprin-α or meprin-β is a competitive inhibitor.

23. The method of claim 21, wherein said inhibitor is a small molecule.

24. A method of inhibiting the release of a full-length Aβ polypeptide from APP or a fragment thereof, comprising:
cleaving said APP or APP fragment by a meprin-α or meprin-β polypeptide.

25. A method for identifying a modulator of the enzymatic production of Aβ from APP or a fragment thereof, comprising:
contacting APP or an APP fragment and meprin-α or meprin-β with a candidate compound; and
monitoring an effect of the candidate compound on production of Aβ.

26. The method of claim 25, wherein said modulator is an inhibitor of Aβ production.

27. The method of claim 26, wherein the effect of the candidate compound on the production of Aβ is monitored by measuring an amount of Aβ formed.

28. The method of claim 25, wherein the effect of the candidate compound on the production of Aβ is monitored by measuring an amount of Aβ1-40 or Aβ1-42 formed.

29. The method of claim 25, wherein the effect of the candidate compound on the production of Aβ is monitored by measuring an amount of Aβ3-40 or Aβ3-42 formed.

30. The method of claim 25, wherein the method is performed in the presence of an α-secretase activity.

31. The method of claim 25, wherein the method is performed in the presence of an γ-secretase activity.

32. The method of claim 25, wherein the method is performed in the presence of an β-secretase activity other than meprin-α or meprin-β activity.

33. The method of claim 32, wherein the method is performed in the presence of BACE1 or BACE2.

34. The method of claim 25, wherein the amount of Aβ formed is reduced by at least about 50%.

35. The method of claim 25, wherein the amount of Aβ formed is reduced by at least about 75%.

36. The method of claim 25, wherein the amount of Aβ formed is reduced by at least about 90%.

37. The method of claim 25, which is performed in a cell-free format.

38. A pharmaceutical composition comprising at least one antagonist of meprin-α or meprin-β optionally in combination with one or more pharmaceutically acceptable diluents or carriers.

39. The pharmaceutical composition according to claim 38, which comprises additionally at least one compound selected from the group consisting of neutron-transmission enhancers, psychotherapeutic drugs, acetylcholine esterase inhibitors, calcium-channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-D-aspartate glutamate receptor antagonists, non-steroidal anti-inflammatory drugs, antioxidants, and serotonergic receptor antagonists.

40. The pharmaceutical composition according to claim 38, which further comprises:
(i) at least one compound, selected from the group consisting of compounds effective against oxidative stress, anti-apoptotic compounds, metal chelators, inhibitors of DNA repair such as pirenzepin and metabolites, 3-amino-1-propanesulfonic acid (3 APS), 1,3-propanedisulfonate (1,3PDS), α-secretase activators, β- and γ-secretase inhibitors, tau proteins, neurotransmitter, β-sheet breakers, attractants for β-amyloid clearing/depleting cellular components, inhibitors of N-terminal truncated amyloid beta including pyroglutamated β-amyloid 3-42, such as inhibitors of glutaminyl cyclase, anti-inflammatory molecules, or cholinesterase inhibitors (ChEIs) such as tacrine, rivastigmine, donepezil, galantamine, niacin or memantine, Ml agonists and other drugs including any amyloid or tau modifying drug and nutritive supplements, and nutritive supplements;
(ii) an antibody according to the present invention; and
(iii) optionally, a pharmaceutically acceptable carrier or a diluent or an excipient.

41. The pharmaceutical composition according to claim 38, which comprises additionally at least one inhibitor of glutaminyl cyclase.

42. A method for reducing the amount of β-amyloid deposits in the central nervous system (CNS) of a mammal comprising:
administering to said mammal an effective amount of an antagonist of meprin-α or meprin-β.

43. A method for the treatment or prevention of amyloidosis comprising:
administering to a subject in need of such treatment an effective amount of an antagonist of meprin-α or meprin-β or a pharmaceutical composition according to claim 38.

44. The method according to claim 43 for the prevention or treatment of a disease selected from the group consisting of Kennedy's disease, duodenal cancer with or without Helicobacter pylori infections, colorectal cancer, Zolliger-Ellison syndrome, gastric cancer with or without Helicobacter pylori infections, pathogenic psychotic conditions, schizophrenia, infertility, neoplasia, inflammatory host responses, cancer, malign metastasis, melanoma, psoriasis, impaired humoral and cell-mediated immune responses, leukocyte adhesion and migration processes in the endothelium, impaired food intake, impaired sleep-wakefulness, impaired homeostatic regulation of energy metabolism, impaired autonomic function, impaired hormonal balance of body fluids, impaired regulation of body fluids, multiple sclerosis, the Guillain-Barré syndrome and chronic inflammatory demyelinizing polyradiculoneuropathy.

45. The method according to claim 43 for the prevention or treatment of a disease selected from the group consisting of mild cognitive impairment, Alzheimer's disease, Familial British Dementia, Familial Danish Dementia, neurodegeneration in Down Syndrome and Huntington's disease.

46. The method of claim 43, wherein said antagonist is a meprin-α antagonist.

47. The method of claim 43, wherein said antagonist is a meprin-β antagonist.

48. The method of claim 43, wherein said antagonist is an inhibitor.

49. The method of claim 43, wherein said antagonist is a competitive inhibitor.

50. The method of claim 43, wherein said antagonist is a small molecule.

51. The method of claim 7, wherein the antagonist of meprin-α or meprin-β comprises a compound selected from the group consisting of actinonin, batimastat, galardin, NNGH, PLG-NHOH, Ro 32-7315, TAPI-0, and captopril.


SEQUENCE LISTING (18 sequences)

SEQ ID NO: 1 - protein, 42 aa, Human [sequence omitted]

SEQ ID NO: 2 - protein, 40 aa, Human
DAEFRHDSGYEVHHQKLVFFAEDVGSNKGAIIGLMVGGVV

SEQ ID NO: 3 - protein, 38 aa, Human
DAEFRHDSGYEVHHQKLVFFAEDVGSNKGAIIGLMVGG

SEQ ID NO: 4 - protein, 40 aa, Human
EFRHDSGYEVHHQKLVFFAEDVGSNKGAIIGLMVGGVVIA

SEQ ID NO: 5 - protein, 38 aa, Human
EFRHDSGYEVHHQKLVFFAEDVGSNKGAIIGLMVGGVV

SEQ ID NO: 6 - protein, 36 aa, Human
EFRHDSGYEVHHQKLVFFAEDVGSNKGAIIGLMVGG

SEQ ID NO: 7 - protein, 30 aa, Human
GLTNIKTEEISEVKMDAEFRHDSGYEVHHQ

SEQ ID NO: 8 - protein, 30 aa, Artificial
  Mutated Sequence
GLTNIKTEEISEVKMDAQFRHDSGYEVHHQ

SEQ ID NO: 9 - protein, 30 aa, Artificial
  Mutated Sequence
GLTNIKTEEISEVKMXAEFRHDSGYEVHHQ

SEQ ID NO: 10 - protein, 30 aa, Artificial
  Mutated Sequence
GLTNIKTEEISEVNLDAEFRHDSGYEVHHQ

SEQ ID NO: 11 - protein, 30 aa, Artificial
  Mutated Sequence
GLTNIKTEEISEVNLDAQFRHDSGYEVHHQ

SEQ ID NO: 12 - protein, 30 aa, Artificial
  Mutated Sequence
GLTNIKTEEISEVNLXAEFRHDSGYEVHHQ

SEQ ID NO: 13 - protein, 746 aa, Human
MAWIRSTCILFFTLLFAHIAAVPIKYLPEENVHDADFGEQKDISEINLAAGLDLFQGDILLQKSRNGLRDPNTRWTFPIPYILADNLGLNAKGAILYAFEMFRLKSCVDFKPYEGESSYIIFQQFDGCWSEVGDQHVGQNISIGQGCAYKAIIEHEILHALGFYHEQSRTDRDDYVNIWWDQILSGYQHNFDTYDDSLITDLNTPYDYESLMHYQPFSFNKNASVPTITAKIPEFNSIIGQRLDFSAIDLERLNRMYNCTTTHTLLDHCTFEKANICGMIQGTRDDTDWAHQDSAQAGEVDHTLLGQCTGAGYFMQFSTSSGSAEEAALLESRILYPKRKQQCLQFFYKMTGSPSDRLVVWVRRDDSTGNVRKLVKVQTFQGDDDHNWKIAHVVLKEEQKFRYLFQGTKGDPQNSTGGIYLDDITLTETPCPTGVWTVRNFSQVLENTSKGDKLQSPRFYNSEGYGFGVTLYPNSRESSGYLRLAFHVCSGENDAILEWPVENRQVIITILDQEPDVRNRMSSSMVFTTSKSHTSPAINDTVIWDRPSRVGTYHTDCNCFRSIDLGWSGFISHQMLKRRSFLKNDDLIIFVDFEDITHLSQTEVPTKGKRLSPQGLILQGQEQQVSEEGSGKAMLEEALPVSLSQGQPSRQKRSVENTGPLEDHNWPQYFRDPCDPNPCQNDGICVNVKGMASCRCISGHAFFYTGERCQAVQVHGSVLGMVIGGTAGVIFLTFSIIAILSQRPRK

SEQ ID NO: 14 - protein, 747 aa, Murine
MLWIQPACLLSLIFSAHIAAVSIKHLLNGSDHDTDVGEQKDIFEINLAAGLNLFQGDILLPRTRNAMRDPSSRWKLPIPYILADNLELNAKGAILHAFEMFRLKSCVDFKPYEGESSYIIFQKLSGCWSMIGDQQVGQNISIGEGCDFKATIEHEILHALGFFHEQSRTDRDDYVNIWWDQIITDYEHNFNTYDDNTITDLNTPYDYESLMHYGPFSFNKNESIPTITTKIPEFNTIIGQLPDFSAIDLIRLNRMYNCTATHTLLDHCDFEKTNVCGMIQGTRDDADWAHGDSSQPEQVDHTLVGQCKGAGYFMFFNTSLGARGEAALLESRILYPKRKQQCLQFFYKMTGSPADRFEVWVRRDDNAGKVRQLAKIQTFQGDSDHNWKIAHVTLNEEKKFRYVFLGTKGDPGNSSGGIYLDDITLTETPCPAGVWTIRNISQILENTVKGDKLVSPRFYNSEGYGVGVTLYPNGRITSNSGFLGLTFHLYSGDNDAILEWPVENRQAIMTILDQEADTRNRMSLTLMFTTSKNQTSSAINGSVIWDRPSKVGVYDKDCDCFRSLDWGWGQAISHQLLKRRNFLKGDSLIIFVDFKDLTHLNRTEVPASARSTMPRGLLLQGQESPALGESSRKAMLEESLPSSLGQRHPSRQKRSVENTGPMEDHNWPQYFRDPCDPNPCQNEGTCVNVKGMASCRCVSGHAFFYAGERCQAMHVHGSLLGLLIGCIAGLIFLTFVTFSTTNGKLRQ

SEQ ID NO: 15 - protein, 701 aa, Human
MDLWNLSWFLFLDALLVISGLATPENFDVDGGMDQDIFDINEGLGLDLFEGDIRLDRAQIRNSIIGEKYRWPHTIPYVLEDSLEMNAKGVILNAFERYRLKTCIDFKPWAGETNYISVFKGSGCWSSVGNRRVGKQELSIGANCDRIATVQHEFLHALGFWHEQSRSDRDDYVRIMWDRILSGREHNFNTYSDDISDSLNVPYDYTSVMHYSKTAFQNGTEPTIVTRISDFEDVIGQRMDFSDSDLLKLNQLYNCSSSLSFMDSCSFELENVCGMIQSSGDNADWQRVSQVPRGPESDHSNMGQCQGSGFFMHFDSSSVNVGATAVLESRTLYPKRGFQCLQFYLYNSGSESDQLNIYIREYSADNVDGNLTLVEEIKEIPTGSWQLYHVTLKVTKKFRVVFEGRKGSGASLGGLSIDDINLSETRCPHHIWHIRNFTQFIGSPNGTLYSPPFYSSKGYAFQIYLNLAHVTNAGIYFHLISGANDDQLQWPCPWQQATMTLLDQNPDIRQRMSNQRSITTDPFMTTDNGNYFWDRPSKVGTVALFSNGTQFRRGGGYGTSAFITHERLKSRDFIKGDDVYILLTVEDISHLNSTQIQLTPAPSVQDLCSKTTCKNDGVCTVRDGKAECRCQSGEDWWYMGERCEKRGSTRDTIVIAVSSTVAVFALMLIITLVSVYCTRKKYRERMSSNRPNLTPQNQHAF

SEQ ID NO: 16 - protein, 704 aa, Murine
MDARHQPWFLVFATFLLVSGLPAPEKFVKDIDGGIDQDIFDINQGLGLDLFEGDIKLEANGKNSIIGDHKRWPHTIPYVLEDSLEMNAKGVILNAFERYRLKTCIDFKPWSGEANYISVFKGSGCWSSVGNIHAGKQELSIGTNCDRIATVQHEFLHALGFWHEQSRADRDDYVIIVWDRIQPGKEHNFNIYNDSVSDSLNVPYDYTSVMHYSKTAFQNGTESTIVTRISEFEDVIGQRMDFSDYDLLKLNQLYNCTSSLSFMDSCDFELENICGMIQSSGDSADWQRVSQVLSGPESDHSKMGQCKDSGFFMHFNTSILNEGATAMLESRLLYPKRGFQCLEFYLYNSGSGNDQLNIYTREYTTGQQGGVLTLQRQIKEVPIGSWQLHYVTLQVTKKFRVVFEGLRGPGTSSGGLSIDDINLSETRCPHHIWHIQNFTQILGGQDTSVYSPPFYSSKGYAFQIYMDLRSSTNVGIYFHLISGANDDQLQWPCPWQQATMTLLDQNPDIRQRMFNQRSITTDPTMTSDNGSYFWDRPSKVGVTDVFPNGTQFSRGIGYGTTVFITRERLKSREFIKGDDIYILLTVEDISHLNSTSAVPDPVPTLAVHNACSEVVCQNGGICVVQDGRAECKCPAGEDWWYMGKRCEKRGSTRDTVIIAVSSTVTVFAVMLIITLVSVYCTRRKYRKKARANTAAMTLENQHAF

SEQ ID NO: 17 - protein, 695 aa, Human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 18 - protein, 695 aa, Murine
MLPSLALLLLAAWTVRALEVPTDGNAGLLAEPQIAMFCGKLNMHMNVQNGKWESDPSGTKTCIGTKEGILQYCQEVYPELQITNVVEANQPVTIQNWCKRGRKQCKTHTHIVIPYRCLVGEFVSDALLVPDKCKFLHQERMDVCETHLHWHTVAKETCSEKSTNLHDYGMLLPCGIDKFRGVEFVCCPLAEESDSVDSADAEEDDSDVWWGGADTDYADGGEDKVVEVAEEEEVADVEEEEADDDEDVEDGDEVEEEAEEPYEEATERTTSTATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHPFGVDSVPANTENEVEPVDARPAADRGLTTRPGSGLTNIKTEEISEVKMDAEFGHDSGFEVRHQKLVFFAEDVGSNKGAIIGLMVGGVVIATVIVITLVMLKKKQYTSIHHGVVEVDAAVTPEERHLSKMQQNGYENPTYKFFEQMQN